(19) United States
(12) Patent Application Publication
(10) Pub. No.: US 2016/0152969 A1
(43) Pub. Date: Jun. 2, 2016

(54) NUCLEIC ACID EXTRACTION DEVICE

(71) Applicant: Seiko Epson Corporation, Tokyo (JP)

(72) Inventors: Masato HANAMURA, Shiojiri (JP); Kotaro IDEGAMI, Chino (JP); Yuji SAITO, Shiojiri (JP); Kiyohito YAMADA, Shiojiri (JP)

(21) Appl. No.: 14/951,789

(22) Filed: Nov. 25, 2015

(30) Foreign Application Priority Data
Nov. 27, 2014 (JP) 2014-239976

(51) Int. Cl. C12N 15/10

(52) U.S. Cl. CPC C12N 15/101

(57) ABSTRACT

A nucleic acid extraction device includes a tube part in which a first plug of an oil, a second plug of a cleansing liquid not miscible with the oil for cleansing a material with adsorbed nucleic acid, a third plug of an oil, a fourth plug of an eluate not miscible with the oil for eluting nucleic acid from the material, and a fifth plug of an oil are provided in this order, and a cover part provided to surround the tube part.

### BACKGROUND

1. Technical Field

The present invention relates to a nucleic acid extraction device.

2. Related Art

Recently, with the development of utilization technologies of genes, medical services utilizing genes for genetic diagnoses, gene therapies, etc. have attracted attention. Further, in the agricultural and livestock fields, many techniques using genes for variety discrimination and breed improvement have been developed. As technologies for utilizing genes, technologies including PCR (Polymerase Chain Reaction) have been widespread. Today, PCR is an essential technology in elucidation of information of biological materials. PCR is a technique of applying a thermal cycle to nucleic acid as a target of amplification (target nucleic acid) and a solution containing a reagent (reaction liquid), and thereby, amplifying the target nucleic acid. As the thermal cycle of PCR, generally, a thermal cycle at two or three phases of temperatures is applied.

On the other hand, for diagnoses of infections represented by influenza in medical settings, currently, simple test kits of immunochromatography or the like are mainly used. However, in the simple test, the accuracy may be insufficient, and application of PCR expected for the higher test accuracy to infection diagnoses is desired. Further, in general outpatient practices etc. in medical institutions, the times taken for tests are restricted to be shorter because the consultation times are restricted. Accordingly, for example, tests of influenza are made in shorter times by simple tests of immunochromatography or the like at the sacrifice of the test accuracy in the present circumstances.

Under the circumstances, in the medical settings, in order to realize tests by PCR expected for the higher test accuracy, it has been necessary to shorten the time taken for reaction. As an apparatus for reaction of PCR in a shorter time, for example, Patent Document 1 (JP-A-2009-136250) discloses a biological specimen reaction apparatus that rotates a biological specimen reaction chip filled with a reaction liquid and a liquid not miscible with the reaction liquid and having lower specific gravity than the reaction liquid about a rotation axis in the horizontal direction, and thereby, moving the reaction liquid and applying a thermal cycle. Further, as one technique of PCR, a method using magnetic beads (Patent Document 2 (JP-A-2009-207459)), a method of performing a thermal cycle of PCR by moving droplets in a temperature-change region on a substrate using the magnetic beads as moving means for the droplets, etc. are disclosed.

As described above, studies on shortening of the time taken for the thermal cycle of PCR have been conducted, however, a technology of shortening a time taken from extraction of nucleic acid as a template from a specimen to the start of PCR has not been sufficiently developed. For example, for PCR, a treatment of extracting nucleic acid (DNA: Deoxyribonucleic Acid and/or RNA: Ribonucleic Acid) as a template from a specimen (blood, nasal cavity mucus, oral cavity mucus, or the like) (hereinafter, simply referred to as “pretreatment”) is necessary, however, even if it is possible to shorten only the time taken for the thermal cycle of PCR, but it is impossible to shorten the time taken for extraction of the nucleic acid (pretreatment), the requirements in the medical settings are not sufficiently fulfilled.

Generally, pretreatment using columns and magnetic beads is performed, and all of dispensing and stirring/centrifugal operations of the specimen are manually performed or an expensive large-scaled apparatus such as an automatic extraction apparatus is required. In either method, time and effort for at least thirty minutes are required for the pretreatment. Therefore, if only the thermal cycle of PCR is performed in a short time (e.g., within 15 minutes), with the time taken for the pretreatment, the total test time from collection of the specimen to obtainment of the test result is taken for about one hour at the shortest in the present circumstances.

Therefore, it has been practically impossible to consistently perform extraction of the nucleic acid (pretreatment) to the thermal cycle of PCR in the setting with restricted consultation time or the like. Such a situation is one of obstacles to widespread use of the test technique by PCR in medical institutions. That is, the time and trouble required for the PCR itself and the pretreatment make widespread use in the medical settings harder though the PCR is the test method with higher sensitivity and higher accuracy than immunochromatography.

### SUMMARY

An advantage of some aspects of the invention is to provide a nucleic acid extraction device that may shorten a time taken for pretreatment for PCR.

A nucleic acid extraction device according to an aspect of the invention includes a tube part in which a first plug of an oil, a second plug of a cleansing liquid not miscible with the oil for cleansing a material with adsorbed nucleic acid, a third plug of an oil, a fourth plug of an eluate not miscible with the oil for eluting nucleic acid from the material, and a fifth plug of an oil are provided in this order, and a cover part provided to surround the tube part.

The cover part may be detachable and may be extendable in a direction in which the tube part extends. The tube part and the cover part may be separated. It is preferable that a distance from an inner cavity surface of the tube part and an outer surface of the cover part is equal to or more than 3 mm. The cover part may be provided with a slit extending in the direction in which the tube part extends. The cover part may be provided with a hole. The cover part may be formed using a deformable material, and a gas may be enclosed in between the tube part and the cover part and contact between the tube part and the cover part may be prevented. The cover part may contain a non-magnetic material selected from metals or alloys.

A nucleic acid extraction device according to another aspect of the invention includes a tube part in which a first plug of an oil, a second plug of a cleansing liquid not miscible with the oil for cleansing a material with adsorbed nucleic acid, a third plug of an oil, a fourth plug of an eluate not miscible with the oil for eluting nucleic acid from the material, and a fifth plug of an oil are provided in this order, wherein a thickness of a side wall of the tube part is equal to or more than 3 mm. The side wall of the tube part may contain a non-magnetic material selected from metals or alloys.

In this specification, “plug” of a liquid refers to a shape in which only the liquid substantially occupies an interior in a tube or in a longitudinal direction of a tube part, and refers to a state in which an internal space of the tube or the tube part is partitioned by the plug. The expression “substantially” here includes existence of a small amount (e.g. a thin film) of another material (liquid or the like) around the plug, i.e., on an inner wall of the tube or the tube part. Further, the tube or the tube part refers to a tubular part and the tube or the tube part refers to a tubular deformable part having a section of an inner cavity in which the liquid may maintain the plug within the tube or the tube part.

### BRIEF DESCRIPTION OF THE DRAWINGS

The invention will be described with reference to the accompanying drawings, wherein like numbers reference like elements.

FIG. 1 schematically shows a major part of a nucleic acid extraction device according to an embodiment.

FIG. 2 schematically shows a major part of a nucleic acid extraction device according to an embodiment.

FIG. 3 schematically shows a major part of a nucleic acid extraction device according to an embodiment.

FIG. 4 schematically shows a nucleic acid extraction device according to an embodiment.

FIG. 5 schematically shows a nucleic acid extraction device according to an embodiment.

FIG. 6 schematically shows a nucleic acid extraction device according to an embodiment.

FIG. 7 schematically shows a nucleic acid extraction device according to an embodiment.

FIG. 8 schematically shows a nucleic acid extraction device according to an embodiment.

FIG. 9A schematically shows a nucleic acid extraction device according to an embodiment. FIG. 9B is a sectional view along a broken line.

FIG. 10A schematically shows a nucleic acid extraction device according to an embodiment. FIG. 10B is a sectional view along a broken line.

FIG. 11A schematically shows a nucleic acid extraction device according to an embodiment. FIG. 11B schematically shows a nucleic acid extraction device according to an embodiment.

FIG. 12 schematically shows a nucleic acid extraction device according to an embodiment.

FIG. 13 schematically shows a major part of a nucleic acid extraction device according to an embodiment.

FIG. 14 schematically shows an example of a nucleic acid extraction kit according to an embodiment.

FIG. 15 schematically shows an example of a nucleic acid extraction kit according to an embodiment.

FIG. 16 is a schematic diagram for explanation of a modified example of a nucleic acid extraction method of an embodiment.

FIG. 17 is a perspective view showing an example of a nucleic acid extraction apparatus according to an embodiment.

FIG. 18 is a perspective view showing an example of a nucleic acid extraction apparatus according to an embodiment.

FIG. 19 is a graph showing results of experimental examples.

FIG. 20 is a graph showing a relationship between elution temperature and DNA yield.

### DESCRIPTION OF EXEMPLARY EMBODIMENTS

As below, several embodiments of the invention will be explained. The embodiments to be described are for explanation of examples of the invention. The invention is not limited to the following embodiments and includes various modified forms embodied in a range in which the subject matter of the invention is not changed. Not all of the configurations to be described are necessarily essential component elements of the invention.

### 1. Nucleic Acid Extraction Device

A nucleic acid extraction device 1000 of the embodiment has a tube part 100, and a first plug 10, a second plug 20, a third plug 30, a fourth plug 40, and a fifth plug 50.

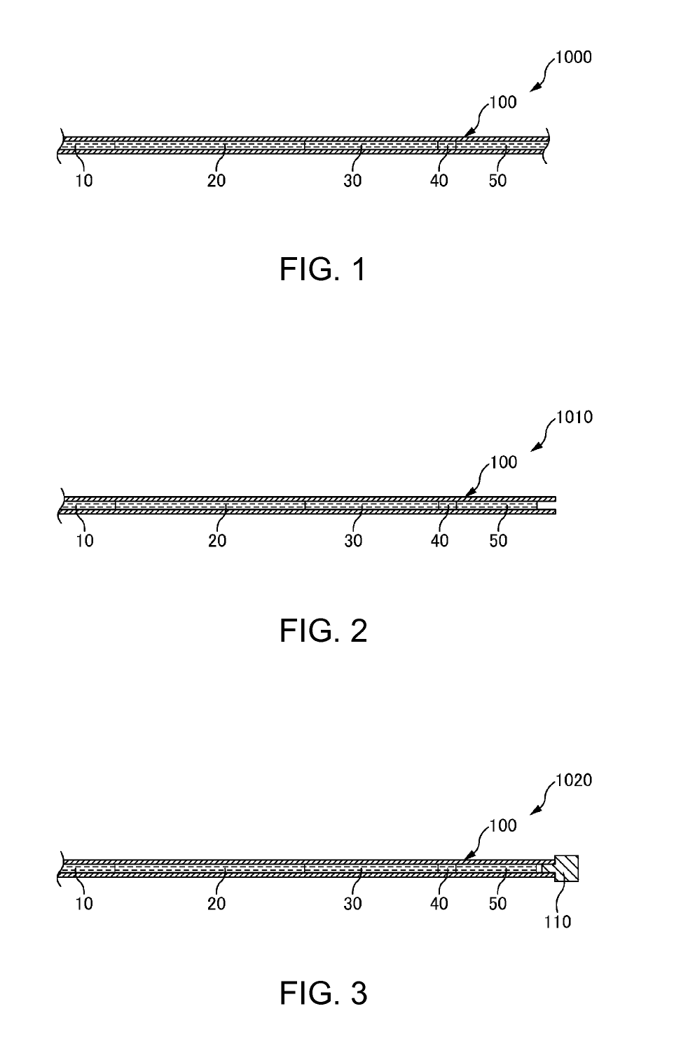

FIG. 1 schematically shows a major part of a nucleic acid extraction device 1000 of the embodiment.

### 1.1. Tube Part

The tube part 100 forms the major part of the nucleic acid extraction device 1000. The nucleic acid extraction device 1000 may include various configurations in addition to the tube part 100. The nucleic acid extraction device 1000 may include e.g. a pipe, a container, a tap, a joint, a pump, a controller, etc. (not shown) connected to the tube part 100.

The tube part 100 is a tubular part having a cavity inside in which a liquid may be passed in the longitudinal direction. The tube part 100 has the longitudinal direction and may bend. Both the size and the shape of the cavity within the tube part 100 are not particularly limited as long as liquids may maintain the plug shape within the tube part 100. Further, the size of the cavity within the tube part 100 and the shape of the section perpendicular to the longitudinal direction may vary along the longitudinal direction of the tube part 100. Whether the liquid may maintain the plug shape within the tube part 100 depends on conditions including the material of the tube part 100 and the kind of the liquid, and accordingly, the shape of the section perpendicular to the longitudinal direction of the tube part 100 is appropriately designed within a range in which the liquid may maintain the plug shape within the tube part 100.

The shape of the section perpendicular to the longitudinal direction of the outer shape of the tube part 100 is not limited. Further, the thickness of the tube part 100 (the length from the side surface of the cavity inside to the surface outside) is not particularly limited. When the section perpendicular to the longitudinal direction of the cavity within the tube part 100 has a circular shape, the inner diameter of the tube part 100 (the diameter of the circle in the section perpendicular to the longitudinal direction of the cavity inside) may be set to be e.g. from 0.5 mm to 3 mm. It is more preferable that the inner diameter of the tube part 100 is within the range because the liquid plug is easily formed in a wide range of the materials of the tube part 100 and the kinds of the liquid.

The material of the tube part 100 is not particularly limited, and may be e.g. glass, polymer, metal, or the like. However, it is more preferable that a material having transparency in visible light such as glass or polymer is selected for the material of the tube part 100 because observation may be made inside (into the cavity) from outside. Further, it is preferable that a material that transmits a magnetic force or a non-magnetic material is selected for the material of the tube part 100 because, when magnetic particles are passed through the tube part 100, this is facilitated by applying a magnetic force from outside of the tube part 100.

Within the tube part 100, the first plug 10 of an oil, the second plug 20 of a first cleansing liquid not miscible with the oil, the third plug 30 of an oil not miscible with the first cleansing liquid, the fourth plug 40 of an eluate not miscible with the oil, and the fifth plug 50 of an oil not miscible with the eluate are provided in the order.

### 1.2. First Plug, Third Plug, Fifth Plug

All of the first plug 10, the third plug 30, and the fifth plug 50 are made of the oils. The oils of the first plug 10, the third plug 30, and the fifth plug 50 may be different kinds of oils from one another. Further, the liquids forming the adjacent plugs to the first plug 10, the second plug 20, the third plug 30, the fourth plug 40, and the fifth plug 50 are selected so as not to be miscible with each other.

As the oils, e.g. silicone oil or mineral oil may be used. Here, the silicone refers to an oligomer or polymer having a siloxane bond as a bone skeleton. In the specification, silicone in a liquid state in the temperature range used for the thermal cycle treatment is particularly referred to as “silicone oil”. Further, in the specification, oil refined from petroleum in a liquid state in the temperature range used for the thermal cycle treatment is referred to as “mineral oil”. These oils are highly stable to heat and products having viscosity of e.g. 5×10³ Nsm⁻² or less are easily available, and preferable for elevating type PCR.

As the silicone oil, dimethyl silicone oil including KF-96L-0.65cs, KF-96L-1cs, KF-96L-2cs, KF-96L-5cs manufactured by Shin-Etsu Silicone, SH200 C FLUID 5 CS manufactured by Dow Corning Toray, or TSF451-5A, TSF451-10 manufactured by Momentive Performance Materials Japan is exemplified. As the mineral oil, mineral oil containing alkane having a carbon number of about 14 to 20 as a major component is exemplified. Namely, n-tetradecane, n-pentadecane, n-hexadecane, n-heptadecane, n-octadecane, n-nonadecane, n-tetracosane are exemplified.

As described above, it is preferable to add an antistatic agent to the oil. As the antistatic agent, e.g. a modified silicone oil may be used. Here, the modified silicone oil refers to a silicone oil having a substituent group. As the antistatic agent, e.g. a carbinol group, an alkylsilyl group, a fluoroalkyl group, a silanol group, or an alkylsilsesquioxy group may preferably be contained as a substituent group. The antistatic agent may have a plurality of kinds of substituent groups, e.g. an alkylsilyl group and an alkylsilsesquioxy group or an alkylsilyl group and a fluoroalkyl group. Further, cyclic siloxane may be used. It is more preferable that the antistatic agent has stability to heat in the temperature range in which the thermal cycle treatment is performed. For example, carbinol-modified silicone oil, KF-6001 manufactured by Shin-Etsu Silicone, BY 16-201, 5562 CARBINOL FLUID manufactured by Dow Corning Toray, and XF42-B0970 manufactured by Momentive Performance Materials Japan are exemplified. The carbinol-modified silicone oil has viscosity of 3×10⁴ Nsm⁻² or more and the viscosity is too high for use for elevating type PCR, however, its volume resistance value is lower than that of dimethyl silicone oil. Accordingly, the carbinol-modified silicone oil is mixed with the dimethyl silicone oil, and thereby, the conductivity of the oil for use may be adjusted. That is, as the additive amount of the carbinol-modified silicone oil is larger, the volume specific resistance becomes smaller. The additive amount is not particularly limited, but it is preferable that the mixed oil has a volume specific resistance value of 5.4×10¹⁰ Ω·cm or less.

The antistatic agent may be a liquid containing a plurality of components or a mixture of a plurality of kinds of liquids. For example, X21-5250 (trimethylsiloxysilicate 50%, cyclopentasiloxane 50%), X21-5616 (trimethylsiloxysilicate 60%, isododecane 40%) manufactured by Shin-Etsu Silicone may be used.

The second plug 20 is provided between the first plug 10 and the third plug 30. Another liquid plug may be provided in a region of the first plug 10 opposite to the second plug 20. It is preferable that there is no air bubbles or other liquids in the first plug 10, however, air bubbles or other liquids may exist as long as particles with adsorbed nucleic acid may pass through the first plug 10. Further, it is preferable that there is no air bubbles or other liquids between the first plug 10 and the second plug 20, however, air bubbles or other liquids may exist as long as particles with adsorbed nucleic acid may pass from the first plug 10 to the second plug 20. Similarly, it is preferable that there is no air bubbles or other liquids between the second plug 20 and the third plug 30, however, air bubbles or other liquids may exist as long as particles with adsorbed nucleic acid may pass from the second plug 20 to the third plug 30.

The fourth plug 40 is provided between the third plug 30 and the fifth plug 50. Another liquid plug may be provided in a region of the fifth plug 50 opposite to the fourth plug 40. It is preferable that there is no air bubbles or other liquids in the third plug 30, however, air bubbles or other liquids may exist as long as particles with adsorbed nucleic acid may pass through the third plug 30. Further, it is preferable that there is no air bubbles or other liquids between the third plug 30 and the fourth plug 40, however, air bubbles or other liquids may exist as long as particles with adsorbed nucleic acid may pass from the third plug 30 to the fourth plug 40. Similarly, it is preferable that there is no air bubbles or other liquids between the fourth plug 40 and the fifth plug 50, however, air bubbles or other liquids may exist as long as particles with adsorbed nucleic acid may pass from the fourth plug 40 to the fifth plug 50. Furthermore, it is preferable that there is no air bubbles or other liquids in the fifth plug 50.

All of the lengths of the first plug 10, the third plug 30, and the fifth plug 50 in the longitudinal direction of the tube part 100 are not particularly limited as long as plugs can be formed. The specific lengths of the first plug 10, the third plug 30, and the fifth plug 50 in the longitudinal direction of the tube part 100 are preferably from 1 mm to 50 mm, and, in order not to make the traveling distance of particles etc. too large, preferably from 1 mm to 30 mm and more preferably from 5 mm to 20 mm. Of them, if the length of the third plug 30 in the longitudinal direction of the tube part 100 is made longer, when the form in which the fourth plug 40 is ejected from the end of the tube part 100 at the fifth plug 50 side is employed, the second plug 20 may be made harder to be ejected. In this case, the specific length of the third plug 30 may be from 10 mm to 50 mm.

The first plug 10 and the fifth plug 50 have a function of preventing matter exchange of the first cleansing liquid (the second plug 20) and the eluate (the fourth plug 40) with outer air such as evaporation and contamination from outside even when at least one end of the tube part 100 is opened. Accordingly, even when at least one end of the tube part 100 is opened to the outside air, the volumes of the first cleansing liquid and the eluate may be kept constant and variations in concentration and contamination of the respective liquids may be suppressed. Thereby, the accuracy of the concentrations of the nucleic acid and various reagents in nucleic acid extraction may be improved.

Further, the third plug 30 has a function of suppressing mixing of the first cleansing liquid (the second plug 20) and the eluate (the fourth plug 40) with each other. Furthermore, if the third plug 30 contains oil having the higher viscosity, when particles etc. are moved at the boundary surface between the first cleansing liquid (the second plug 20) and itself, “swab effect” by the oil may be improved. Thereby, when particles etc. are moved from the plug of the first cleansing liquid of the second plug 20 to the third plug 30 of the oil, the water-soluble components attached to the particles etc. are harder to be taken into the third plug 30 (oil).

### 1.3. Second Plug

The second plug 20 is provided in a position between the first plug 10 and the third plug 30 within the tube part 100. The second plug 20 consists of the first cleansing liquid. The first cleansing liquid is a liquid not miscible with the oil forming the first plug 10 or the oil forming the third plug 30. The first cleansing liquid includes water or a buffer solution having a solute concentration of 10 mM or less, preferably 7 mM or less, and more preferably 5 mM or less. The composition of the buffer solution is not particularly limited and e.g. Tris-HCL buffer solution or the like may be exemplified, and may contain EDTA (ethylenediamine tetraacetic acid) or the like. The above described first cleansing liquid may efficiently cleanse the particles etc. with adsorbed nucleic acid.

The volume of the second plug 20 is not particularly limited and may be appropriately set with the amount of particles etc. with adsorbed nucleic acid as an index. For example, when the volume of the particles etc. is 0.5 μL, the volume of the second plug 20 is sufficiently 10 μL or more, preferably from 20 μL to 50 μL, and more preferably from 20 μL to 30 μL. If the volume of the second plug 20 is within the range, when the volume of the particles etc. is 0.5 μL, the particles etc. may be sufficiently cleansed. Note that, for cleansing of the particles etc., it is preferable that the volume of the second plug 20 is larger, however, the volume may be appropriately set in consideration of the length and the thickness of the tube part 100 and the dependent length of the second plug 20 in the longitudinal direction of the tube part 100 etc.

The second plug 20 may be divided by oil plugs and includes a plurality of plugs. When the second plug 20 includes a plurality of plugs divided by oil plugs, a plurality of plugs of the first cleansing liquid are formed. Accordingly, when the second plug 20 is divided by the oil plugs, it is preferable that an object to be cleansed is a water-soluble material because the concentration of the water-soluble material reaching by the divided first cleansing liquid is smaller than the concentration of the water-soluble material reaching by the non-divided first cleansing liquid having the same volume. The number of division of the second plug 20 is arbitrary. When the object to be cleansed is a water-soluble material, for example, if the material is divided in two with equal volumes, the concentration of the water-soluble material may be reduced to one fourth of the concentration without division according to calculations. The number of division of the second plug 20 may be appropriately set in consideration of e.g. the length of the tube part 100, the object to be cleansed, etc.

### 1.4. Fourth Plug

The fourth plug 40 is provided in a position between the third plug 30 and the fifth plug 50 within the tube part 100. The fourth plug 40 consists of the eluate.

The eluate refers to a liquid that desorbs nucleic acid adsorbed to the particles etc. from the particles and elutes the nucleic acid in the liquid. The eluate includes e.g. sterilized water, distilled water, purified water such as ion exchanged water or a water solution formed by dissolving at least one of enzyme, dNTP, probe, primer, and buffer in the water. The eluate is the liquid not miscible with the oil forming the third plug 30 and the oil forming the fifth plug 50.

If the eluate is water or a water solution, the particles etc. with adsorbed nucleic acid are immersed in the eluate, and the nucleic acid adsorbed to the particles etc. may be extricated (eluted). Or, if the water solution formed by dissolving at least one of enzyme, dNTP, probe, primer, and buffer is selected in the eluate, the nucleic acid adsorbed to the particles etc. may be extricated (eluted) and part or all of the components necessary for the reaction liquid of PCR may be contained in the eluate, and thereby, time and effort for preparation of the reaction liquid of PCR using the eluate may be further saved. The concentration when at least one of enzyme, dNTP, probe, primer, and buffer is dissolved in the eluate of the fourth plug 40 is not particularly limited, and may be set according to the reaction liquid of PCR to be prepared.

Note that, here, dNTP refers to four kinds of deoxynucleotide triphosphate (mixture of dATP (Deoxyadenosine triphosphate), dCTP (Deoxycytidine triphosphate), dGTP (Deoxyguanosine triphosphate), and dTTP (Thymidine triphosphate)).

The volume of the fourth plug 40 is not particularly limited and may be appropriately set with the amount of particles etc. with adsorbed nucleic acid as an index. For example, when the volume of the particles etc. is 0.5 μL, the volume of the fourth plug 40 is sufficiently 0.5 μL, or more, preferably from 0.8 μL to 5 μL, and more preferably from 1 μL to 3 μL. If the volume of the fourth plug 40 is within the range, when the volume of the particles etc. is 0.5 μL, elution of nucleic acid from the particles etc. may be sufficiently performed. Note that, for elution of nucleic acid from the particles etc., it is preferable that the volume of the fourth plug 40 maybe appropriately set in consideration of the length and the thickness of the tube part 100 and promptness of the thermal cycle of PCR so that the heat capacity of the reaction liquid may not be too large.

### 1.5. Advantages

The nucleic acid extraction device 1000 of the embodiment has the tube part 100 in which the first cleansing liquid and the eluate are provided in plug shapes. Accordingly, the particles etc. with adsorbed nucleic acid are introduced from the first plug 10 side into the tube part 100 and moved to the fourth plug 40, and thereby, the extraction of the nucleic acid may be easily performed in an extremely short time. More specifically, the particles etc. with adsorbed nucleic acid may be introduced from the first plug 10 side of the tube part 100, passed through the oil of the first plug 10, cleansed by the first cleansing liquid of the second plug 20, and passed through the oil of the third plug 30, and the nucleic acid may be desorbed from the particles etc. in the eluate of the fourth plug 40. Namely, the nucleic acid extraction device 1000 of the embodiment moves the particles etc. with adsorbed nucleic acid within the tube part 100, and thereby, the eluate containing nucleic acid with higher purity may be obtained. Therefore, according to the nucleic acid extraction device 1000, time and effort required for the pretreatment for PCR may be significantly reduced.

### 1.6. Configuration etc. of Nucleic Acid Extraction Device

The nucleic acid extraction device of the embodiment has the tube part 100, the first plug 10, the second plug 20, the third plug 30, the fourth plug 40, and the fifth plug 50, and may include configurations for adding other functions. The nucleic acid extraction device of the embodiment may include combinations of configurations to be described and modified forms of the respective configurations.

### 1.6.1. End of Tube Part

FIG. 2 schematically shows a nucleic acid extraction device 1010 as a kind of modified example of the nucleic acid extraction device. In the nucleic acid extraction device of the embodiment, for example, the end of the tube part 100 at the fifth plug 50 side may be opened. That is, as shown in FIG. 2, in the nucleic acid extraction device 1010, the end of the tube part 100 at the fifth plug 50 side is opened. According to the nucleic acid extraction device 1010, the fifth plug 50 and the fourth plug 40 may be sequentially ejected by application of pressure from the first plug 10 side of the tube part 100 into the tube part 100. Thereby, the eluate containing target nucleic acid (the fourth plug 40) may be easily dispensed to e.g. a reaction container for PCR or the like.

### 1.6.2. Tap

FIG. 3 schematically shows a nucleic acid extraction device 1020 as a kind of modified example of the nucleic acid extraction device. As shown in the drawing, the nucleic acid extraction device of the embodiment may further have e.g. a detachable tap 110 that seals the end of the tube part 100 at the fifth plug 50 side. The tap 110 may be formed using e.g. rubber, elastomer, polymer, or the like. When the tube part 100 is sealed by the tap 110, the tap 110 may be in contact with the fifth plug 50 or a gas such as air may be placed between the fifth plug 50 and the tap 110. The tap 110 is detachable and its mechanism is not particularly limited. In the example of FIG. 3, a form in which a part of the tap 110 is inserted into the tube part 100 and fixed is shown, however, the tap 110 may have a cap shape.

In the nucleic acid extraction device 1020, when the tap 110 is detached, the end of the tube part 100 at the fifth plug 50 side is opened and the form of the above described nucleic acid extraction device 1010 in FIG. 2 is obtained, and, using the nucleic acid extraction device 1020, the eluate containing target nucleic acid (the fourth plug 40) may be easily dispensed to e.g. a reaction container for PCR or the like. Or, when the end of the tube part 100 at the fifth plug 50 side is sealed by the tap 110 (as shown in FIG. 3), an advantage of suppressing the movements of the respective plugs within the tube part 100 is obtained, and thereby, for example, when the particles etc. are moved within the tube part 100, movements of the plugs with the movements of the particles etc. may be suppressed.

### 1.6.3. Container

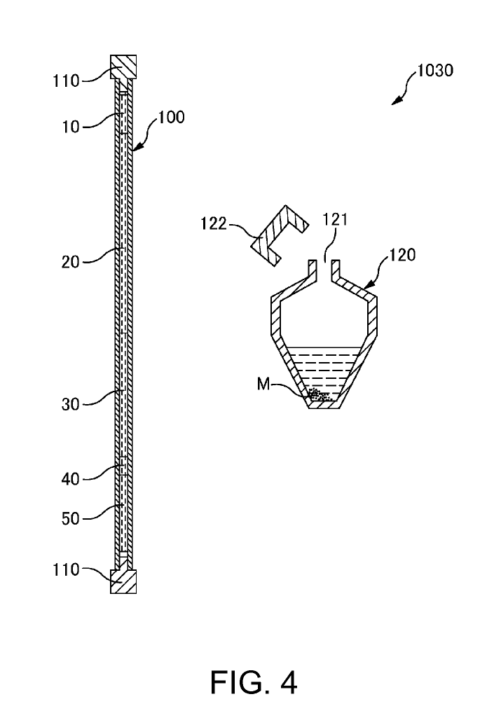

FIG. 4 schematically shows a nucleic acid extraction device 1030 as an example of the configuration of the nucleic acid extraction device. As exemplified in FIG. 4, the nucleic acid extraction device 1030 further has a detachable container 120 that may be connected to the end of the tube part 100 at the first plug 10 side with interiors communicating with each other.

The container 120 may be an independent member. The container 120 may contain a liquid inside. The container 120 has an opening 121 through which a liquid or a solid may be put in and out. Further, in the example of FIG. 4, the opening 121 of the container 120 is connected to the end of the tube part 100 at the first plug 10 side with interiors communicating with each other. Furthermore, the container 120 may have a plurality of the openings 121 and one of the openings 121 may be connected to the end of the tube part 100 at the first plug 10 side with interiors communicating with each other.

The inner volume of the container 120 is not particularly limited, and may be e.g. from 0.1 mL to 100 mL. The opening 121 of the container 120 may have a structure that can be sealed by a lid 122 as appropriate. The material of the container 120 is not particularly limited, and may be polymer, metal, or the like.

The opening 121 of the container 120 may be connected to the end of the tube part 100 at the first plug 10 side, however, the connection between the container 120 and the tube part 100 is not particularly limited as long as its contents do not leak. When the container 120 and the tube part 100 are connected, the interior of the container 120 and the interior of the tube part 100 may communicate with each other. Further, the container 120 may be detached from the tube part 100 as appropriate.

According to the nucleic acid extraction device 1030, the container 120 is provided, and thereby, within the container 120, e.g. particles etc. an adsorption liquid, and a specimen may be contained and nucleic acid may be adsorbed to the particles etc. Then, the container 120 is connected to the end of the tube part 100 at the first plug 10 side, and thereby, the particles etc. may be easily introduced into the tube part 100 from the first plug 10 side of the tube part 100.

The adsorption liquid refers to a liquid in which particles (magnetic particles M) are allowed to adsorb nucleic acid, e.g., a water solution containing a chaotropic agent. The adsorption liquid may contain a chelating agent, a surface-active agent, or the like. Specifically, in the adsorption liquid, ethylenediaminetetraacetic acid disodium salt or dihydrate thereof may be dissolved or the liquid may contain polyoxyethylene sorbitan monolaurate or the like.

Here, the chaotropic agent refers to a material that reduces interaction between water molecules, and thereby, destabilizes the structures of the water molecules. Specifically, the agent includes guanidium ions, urea, and iodide ions. When the chaotropic agent exists in water, the nucleic acid in the water is thermodynamically more favorable inexistence adsorbed to the solid than inexistence surrounded by water molecules, and thereby, adsorbed to the surfaces of particles etc. The material that may generate the chaotropic agent in water includes guanidinium hydrochloride and sodium iodide.

The container 120 may be shaken without connection to the tube part 100, and the liquid within the container 120 may be sufficiently stirred. Thereby, the nucleic acid may be promptly adsorbed to the particles etc. The container 120 may have the lid 122 that seals the opening 121. Further, the amount of the specimen introduced into the container 120 and the volume of the liquid within the tube part 100 (particularly, the fourth plug 40) are appropriately changed, and thereby, the nucleic acid in the specimen may be quantitatively concentrated in the eluate of the fourth plug 40.

When a material of rubber, elastomer, polymer, or the like having flexibility is selected for the material of the container 120, the container 120 connected to the tube part 100 is deformed, and thereby, the interior of the tube part 100 may be pressurized. In this manner, when the eluate of the fourth plug 40 is ejected from the end of the tube part 100 at the fifth plug 50 side, pressure is easily applied from the first plug 10 side of the tube part 100. Thereby, the eluate may be dispensed to e.g. the reaction container for PCR or the like.

### 1.6.4. Reservoir Part

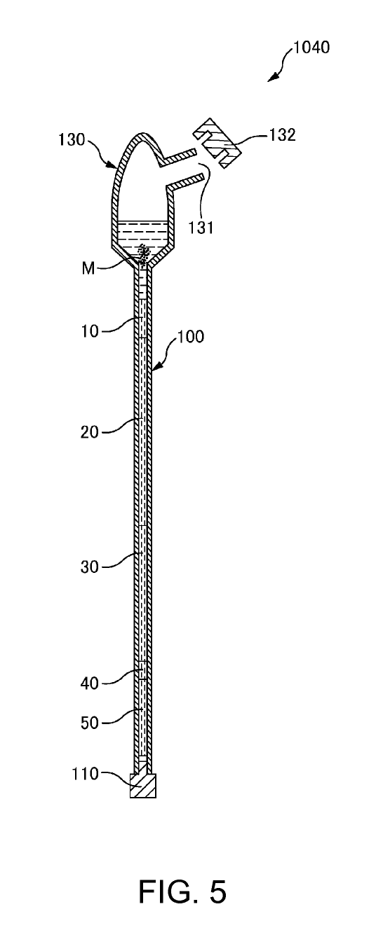

FIG. 5 schematically shows a nucleic acid extraction device 1040 as an example of the configuration of the nucleic acid extraction device. As exemplified in FIG. 5, in the nucleic acid extraction device 1040, a reservoir part 130 communicating with the tube part 100 is formed on the end of the tube part 100 at the first plug 10 side. The interior of the reservoir part 130 and the interior of the tube part 100 communicate with each other.

The reservoir part 130 may contain a liquid inside. The reservoir part 130 has an opening 131 from which a material is introduced inside from outside. The position in which the opening 131 in the reservoir part 130 is formed is not particularly limited. The reservoir part 130 may have a plurality of the openings 131. The inner volume of the reservoir part 130 is not particularly limited, and may be e.g. from 0.1 mL to 100 mL. The material of the reservoir part 130 is not particularly limited, and may be polymer, metal, or the like and may be the same as the material of the tube part 100.

According to the nucleic acid extraction device 1040, the reservoir part 130 is provided, and thereby, within the reservoir part 130, e.g. particles etc. an adsorption liquid, and a specimen may be contained and nucleic acid may be adsorbed to the particles etc. Then, the particles etc. may be easily introduced into the tube part 100 from the first plug 10 side of the tube part 100.

Further, the reservoir part 130 may be shaken with the tube part 100, and the liquid within the reservoir part 130 may be sufficiently stirred. Thereby, the nucleic acid may be promptly adsorbed to the particles etc. Furthermore, the amount of the specimen introduced into the reservoir part 130 and the volume of the liquid within the tube part 100 are appropriately changed, and thereby, the nucleic acid in the specimen may be quantitatively concentrated in the eluate.

When the reservoir part 130 is provided like the nucleic acid extraction device 1040, a detachable lid 132 sealing the opening 131 of the reservoir part 130 may be further provided. When a material of rubber, elastomer, polymer, or the like having flexibility is selected for the material of the reservoir part 130, the reservoir part 130 with the lid 132 attached thereto is deformed, and thereby, the interior of the tube part 100 may be pressurized.

In this manner, when the eluate of the fourth plug 40 in which the nucleic acid is eluted is ejected from the end of the tube part 100 at the fifth plug 50 side, pressure may be easily applied from the first plug 10 side of the tube part 100. Thereby, the step of introducing the specimen into the container 120 to the step of dispensing the eluate to e.g. the reaction container for PCR or the like may be performed. Further, when the lid 132 is attached, liquid leakage when the reservoir part 130 is shaken with the tube part 100 may be suppressed, and the efficiency of the adsorption of the nucleic acid to the particles etc. may be further improved.

### 1.6.5. Structure for Transportation and Storage

When the nucleic acid extraction device is transported and stored, a hand wearing a charged nitrile glove touches the tube part and an electric field is generated between the aqueous solution and the tube. In this case, for example, when the aqueous solution is pushed out of the tube as will be described later, the aqueous solution may be attracted and attached to the inner wall of the tube, only the oil may be pushed out, the aqueous solution may not move and the plug may be split, or the aqueous solution may repel against the inner wall of the tube, and the droplets of the aqueous solution may float in the oil. The aqueous solution split or turned into droplets may move in the oil due to the electrostatic action and mix with the plugs of the other pretreatment reagents. Then, the composition of the mixed aqueous solutions of the plugs may change and the functions of the respective plugs may be lost.

Accordingly, it is preferable that the nucleic acid extraction device has a structure that may prevent charging of the oil and the aqueous solution within the tube part at transportation and storage, and it is preferable that the device has a structure with which a charged material including the hand wearing the charged nitrile glove may not come close to the oil and the aqueous solution within the tube part. Note that, in the specification, “prevent charging” does not refer to necessarily eliminating charging to 100%, but the generated charge may be reduced to the degree at which the tube functions without any difficulty.

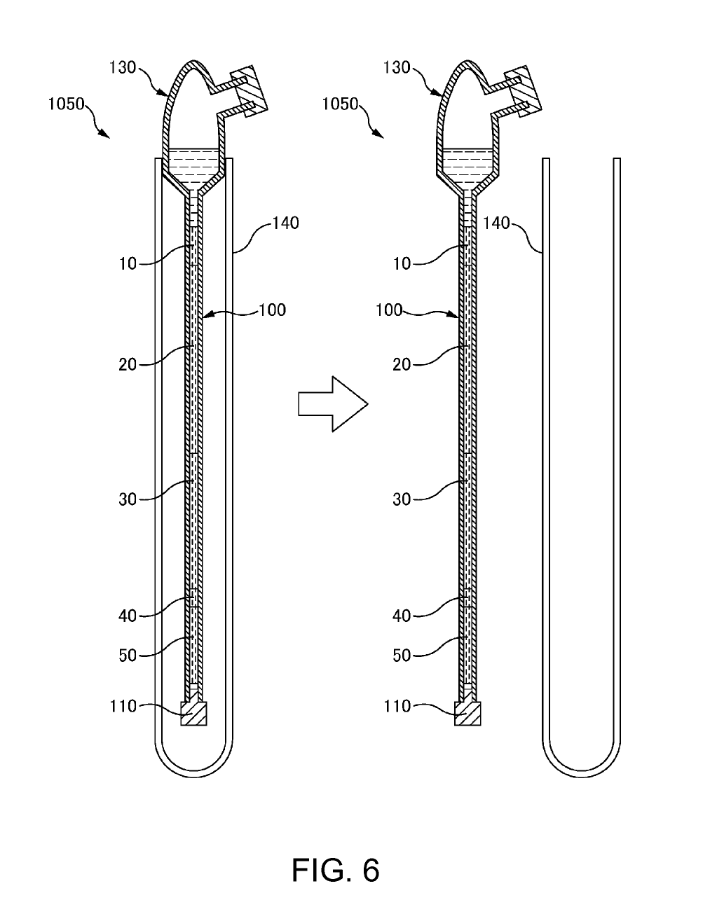

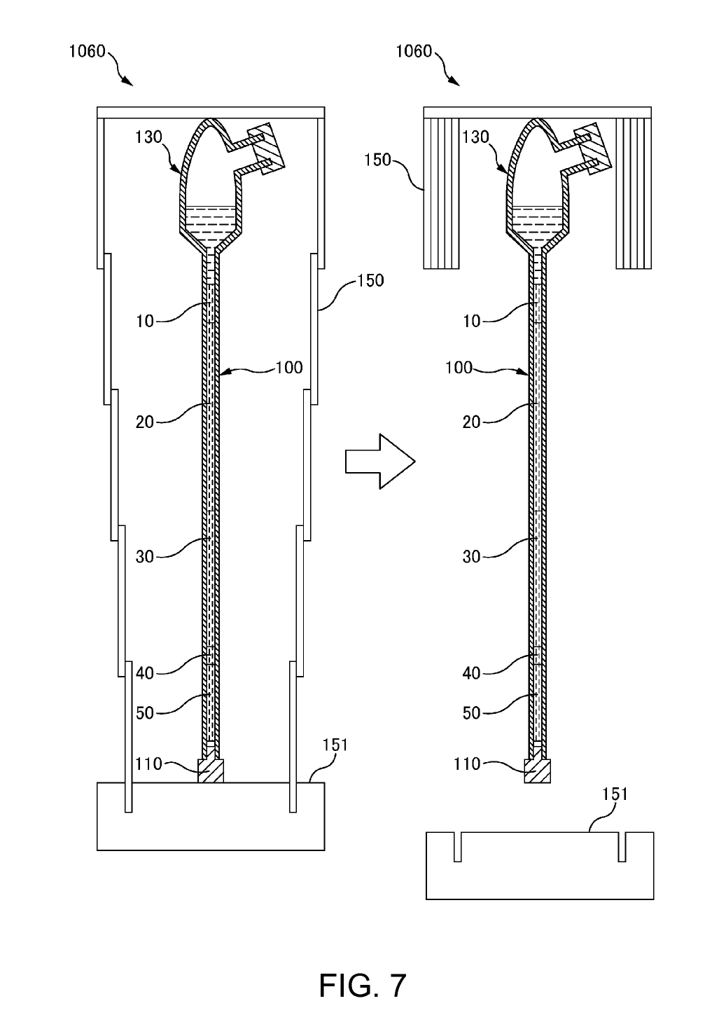

In order that the charged material including the hand wearing the charged nitrile glove may not come close to the oil or the aqueous solution within the tube part, for example, a cover part may be provided to surround the tube part. FIG. 6 schematically shows a nucleic acid extraction device 1050 to which a cap 140 as a detachable cover part that can cover the tube part 100 is attached and the device from which the cap 140 is detached. The material of the cap 140 includes e.g. non-magnetic metal, glass, plastic, rubber, and stone. When the nucleic acid extraction device 1040 is used for nucleic acid extraction, the cap 140 may be removed and a permanent magnet 410, which will be described later, may be brought closer to the tube part 100.

FIG. 7 schematically shows a nucleic acid extraction device 1060 to which an extendable cap 150 with a lid 151 as cover part that can cover the tube part 100 is attached, and the device with the extendable cap 150 contracted. The extendable cap 150 may have any structure that may extend and contract in directions in which the tube part 100 extends, e.g. a bellows structure. When the nucleic acid extraction device 1060 is used for nucleic acid extraction, the lid 151 is removed and the extendable cap 150 is contracted in the extension direction of the tube part 100, and thereby, the tube part 100 is exposed. The extendable cap 150 may be contracted by hand, or a device insertion opening having a smaller opening diameter than the outer diameter of the extendable cap 150 may be provided in the nucleic acid extraction apparatus and the nucleic acid extraction device 1060 may be pressed into the device insertion opening, and thereby, the extendable cap 150 may be contracted and the permanent magnet 410 to be described later may be brought closer to the tube part 100.

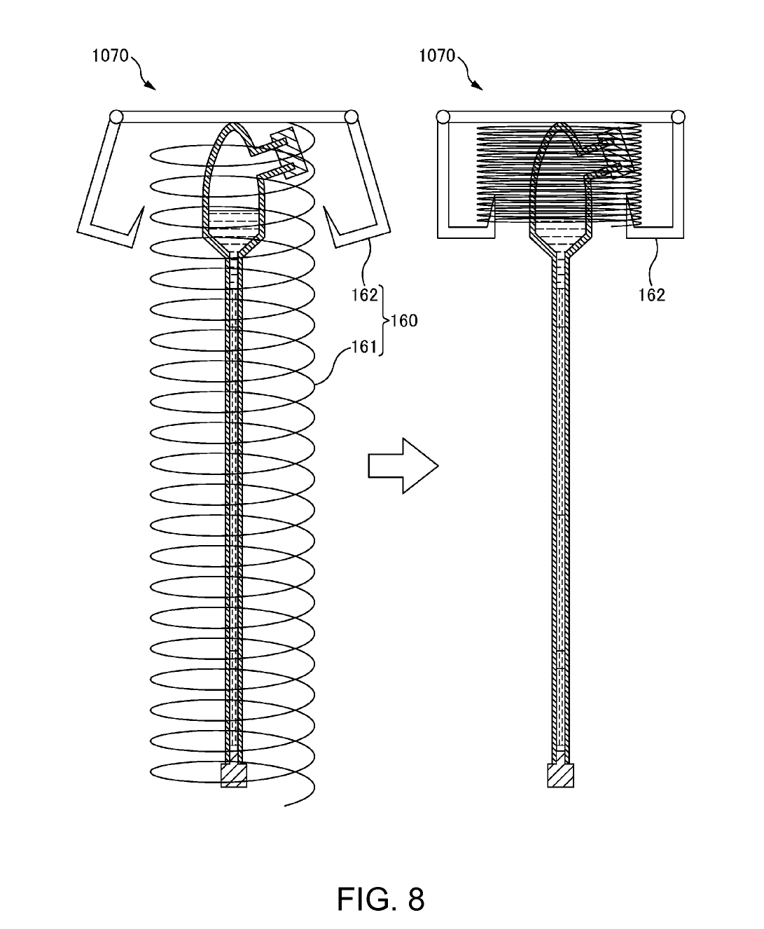

FIG. 8 schematically shows a nucleic acid extraction device 1070 to which a cover 160 having a spring 161 and a holding portion 162 of the spring 161 as an extendable cover part that can cover the tube part 100 is attached, and the device with the spring 161 contracted. When the spring 161 easily swings in the horizontal directions, a support rod for fixing the spring 161 to a predetermined position may be provided inside of the spring 161. When the nucleic acid extraction device 1070 is used for nucleic acid extraction, the spring 161 is contracted by hand in the extension direction of the tube part 100, the tube part 100 is exposed, and the contracted spring 161 is fixed to the holding portion 162. A device insertion opening having a smaller opening diameter than the outer diameter of the spring 161 may be provided in the nucleic acid extraction apparatus, the device may be pressed into the device insertion opening, and thereby, the spring may be contracted and the tube part 100 may be exposed, and the permanent magnet 410 to be described later may be brought closer to the tube part 100.

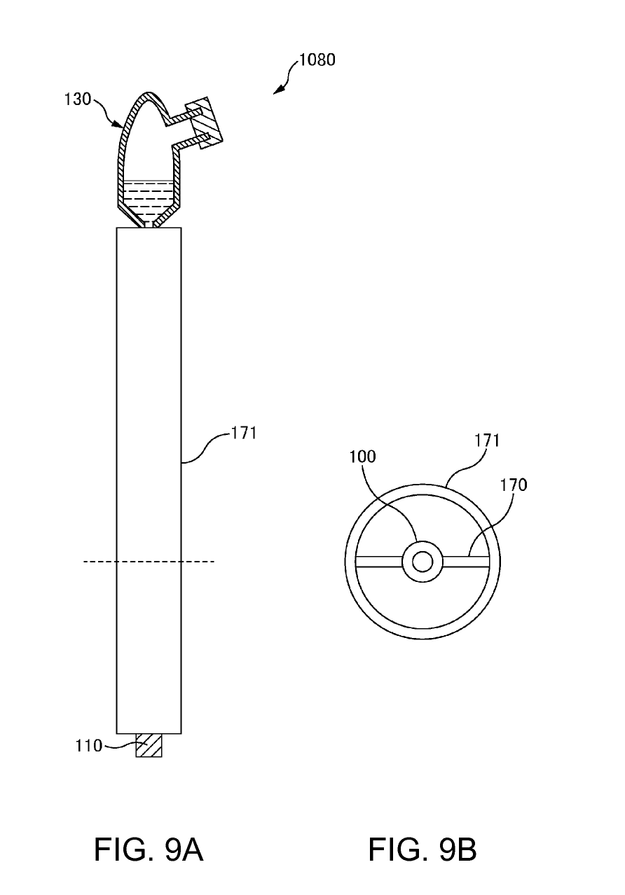

FIGS. 9A and 9B schematically show a nucleic acid extraction device 1080 including a cover part 171 formed via a supporting portion 170 from the tube part 100. FIG. 9B is a sectional view along a broken line in FIG. 9A. When the nucleic acid extraction device 1080 is used for nucleic acid extraction, the permanent magnet 410 may be brought close from the outside of the cover part 171.

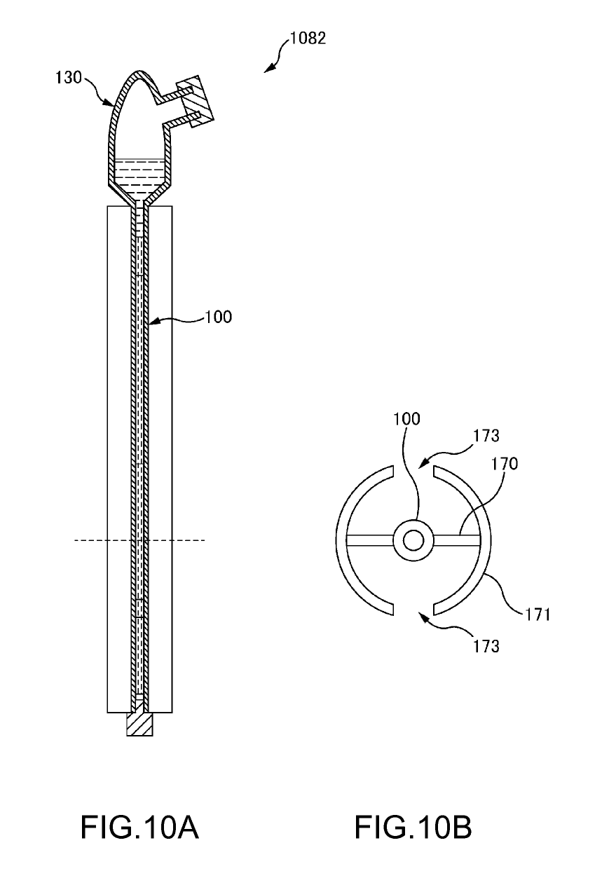

FIGS. 10A and 10B schematically show a nucleic acid extraction device 1082 in which slits 173 extending in the extension direction of the tube part 100 are provided in the cover part 171. FIG. 10B is a sectional view along a broken line in FIG. 10A. When the nucleic acid extraction device 1082 is used for nucleic acid extraction, the permanent magnet 410 to be described later may be inserted into the slits 173 and brought closer to the tube part 100.

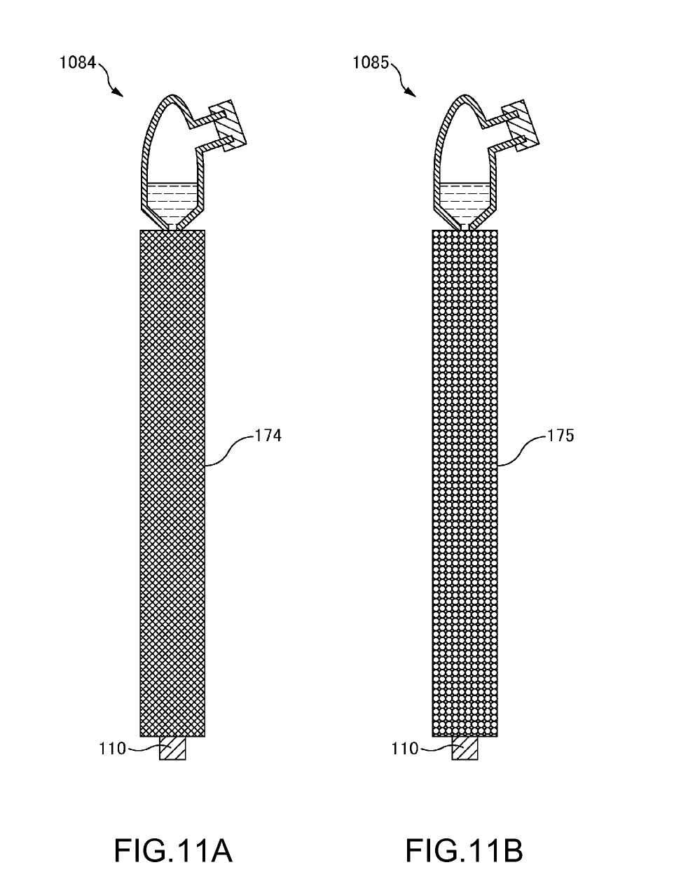

FIG. 11A schematically shows a nucleic acid extraction device 1084 having a cover part 174 in a net-like structure. FIG. 11B schematically shows a nucleic acid extraction device 1085 having a cover part 175 with holes in a polka-dot pattern. One or more holes are formed in the cover part of the nucleic acid extraction device 1084, 1085, however, the size of the hole is smaller than the size of the human finger, and the finger of the hand of the person grasping the nucleic acid extraction device 1084, 1085 is prevented from passing through the cover part to come closer to the tube part 100.

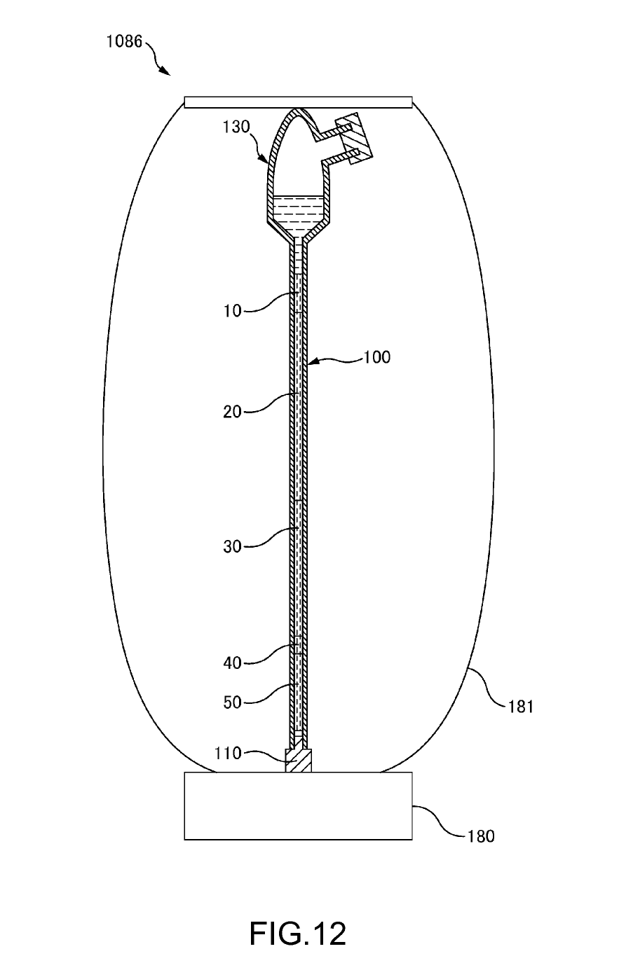

FIG. 12 schematically shows a nucleic acid extraction device 1086 put in a bag 181 with a fastener 180. It is preferable to fill and expand the bag 181 with a gas such as nitrogen to the degree at which the hand does not come close to the inner cavity surface of the tube part 100 within 3 mm so that, when the nucleic acid extraction device 1086 is transported, if the bag 181 is grasped by the hand wearing the charged nitrile glove, attachment of the cover part to the tube part may be prevented. The material of the bag 181 is not particularly limited as long as the material is deformable and e.g. a plastic bag may be used. When the nucleic acid extraction device 1086 is used for nucleic acid extraction, the bag 181 may be broken to expose the tube part 100. Thereby, the permanent magnet 410 may be brought closer to the tube part 100. The bag 181 may have a structure that can be easily broken by pulling the fastener 180.

The thickness of the side wall of the tube part of the nucleic acid extraction device may be made larger so that the charged material including the hand wearing the charged nitrile glove may not come closer to the inner cavity of the tube part. Thereby, even when the charged hand or the like touches the tube part, charging of the oil and the aqueous solution within the tube part may be prevented. It is preferable that the thickness of the side wall of the tube part is from 3 mm to 9.5 mm as long as the thickness may be a thickness that can prevent charging of the oil and the aqueous solution within the tube part and by which the operation using the permanent magnet to be described later may be performed via the side wall of the tube part.

A metal or an alloy of a non-magnetic conducting material is buried in the side wall of the tube part, and thereby, the effect of preventing charging the oil and the aqueous solution within the tube part may be further improved. For example, a spiral copper wire may be buried.

In the above described manner, charging the oil and the aqueous solution within the tube part is prevented, and thereby, the positions of the respective plugs in the tube part may be stably maintained. Further, charging of the nucleic acid extraction device is prevented, and thereby, nucleic acid extraction using the nucleic acid extraction device may be easily automated.

### 1.6.6. Sixth Plug and Seventh Plug

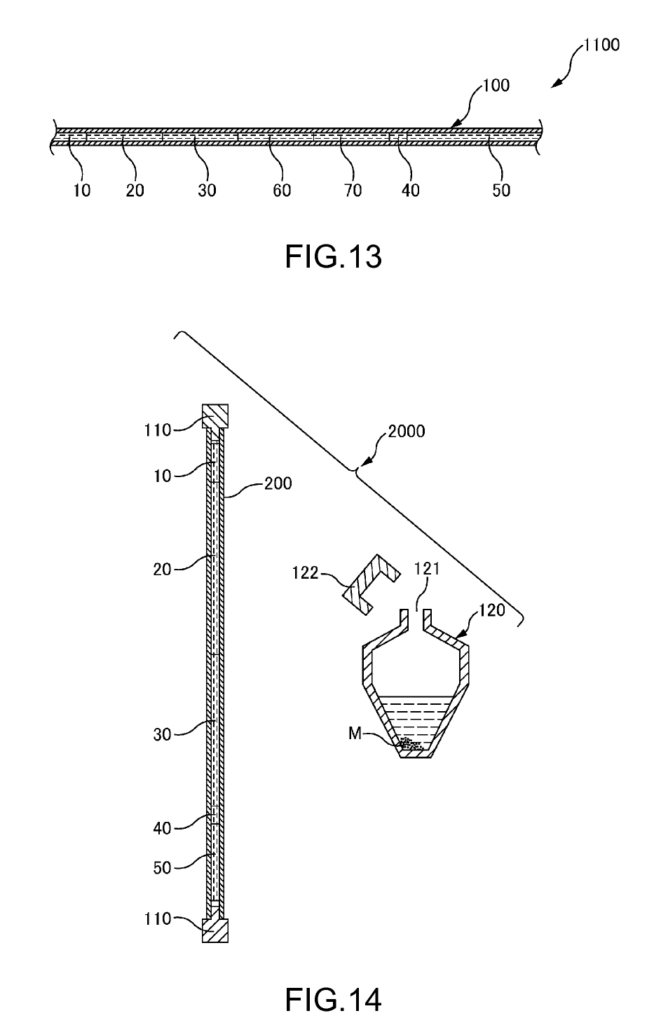

The nucleic acid extraction device of the embodiment may have a sixth plug and a seventh plug inside of the tube part. FIG. 13 schematically shows a nucleic acid extraction device 1100 having a sixth plug 60 and a seventh plug 70 inside of the tube part 100.

The nucleic acid extraction device 1100 has a configuration in which the sixth plug 60 of a second cleansing liquid not miscible with oil and the seventh plug 70 of an oil are added between the third plug 30 and the fourth plug 40 inside of the tube part 100 of the above described nucleic acid extraction device sequentially from the third plug 30.

The sixth plug 60 is provided in the position of the third plug 30 to the opposite side to the second plug 20 within the tube part 100. The sixth plug 60 consists of the second cleansing liquid. The second cleansing liquid is a liquid not miscible with the oil forming the third plug 30 or the oil forming the seventh plug 70. The second cleansing liquid includes water or a buffer solution having solute concentration of 10 mM or less, preferably 7 mM or less, and more preferably 5 mM or less. The composition of the buffer solution is not particularly limited and e.g. Tris-HCL buffer solution or the like may be exemplified, and may contain EDTA (ethylenediamine tetraacetic acid) or the like. The composition of the second cleansing liquid may be the same as or different from that of the first cleansing liquid.

The volume of the sixth plug 60 is not particularly limited and may be appropriately set with the amount of particles etc. with adsorbed nucleic acid as an index. For example, when the volume of the particles etc. is 0.5 μL, the volume of the sixth plug 60 is sufficiently 10 μL or more, preferably from 20 μL to 50 μL, and more preferably from 20 μL to 30 μL. If the volume of the sixth plug 60 is within the range, when the volume of the particles etc. is 0.5 μL, the particles etc. may be sufficiently cleansed. Note that, for cleansing of the particles etc., it is preferable that the volume of the sixth plug 60 is larger, however, the volume may be appropriately set in consideration of the length and the thickness of the tube part 100 and the dependent length of the sixth plug 60 in the longitudinal direction of the tube part 100, etc.

The sixth plug 60 may be divided by oil plugs and includes a plurality of plugs. When the sixth plug 60 includes a plurality of plugs divided by oil plugs, a plurality of plugs of the second cleansing liquid are formed. Accordingly, when the sixth plug 60 is divided by the oil plugs, it is preferable that an object to be cleansed is a water-soluble material because the concentration of the water-soluble material reaching by the divided second cleansing liquid is smaller than the concentration of the water-soluble material reaching by the non-divided second cleansing liquid having the same volume. The number of division of the sixth plug 60 is arbitrary. When the object to be cleansed is a water-soluble material, for example, if the material is divided in two with equal volumes, the concentration of the water-soluble material may be reduced to one fourth of the concentration without division according to calculations. The number of division of the sixth plug 60 may be appropriately set in consideration of e.g. the length of the tube part 100, the object to be cleansed, etc. Note that the first cleansing liquid of the second plug 20 and the second cleansing liquid of the sixth plug 60 may be made the same, the same advantage as that when the second plug 20 is divided in the nucleic acid extraction device without the above described sixth plug 60 and seventh plug 70 may be obtained.

The seventh plug 70 consists of the oil not miscible with the liquids of the adjacent sixth plug 60 and the fourth plug 40. The oil of the seventh plug 70 may be a different kind of oil from the oils of the first plug 10, the third plug 30, and the fifth plug 50. The oil may be the same as those exemplified in the first plug 10 etc.

It is preferable that there is no air bubbles or other liquids in the seventh plug 70, however, air bubbles or other liquids may exist as long as particles etc. with adsorbed nucleic acid may pass through the seventh plug 70. Further, it is preferable that there is no air bubbles or other liquids between the seventh plug 70 and the adjacent fourth plug 40 and sixth plug 60, however, air bubbles or other liquids may exist as long as particles etc. with adsorbed nucleic acid may move within the tube part 100. Note that it is preferable that there is no air bubbles or other liquids in the seventh plug 70.

The length of the seventh plug 70 in the longitudinal direction of the tube part 100 is not particularly limited as long as the plug can be formed. The specific length of the seventh plug 70 in the longitudinal direction of the tube part 100 is from 1 mm to 50 mm, and, in order not to make the traveling distances of particles etc. too large, preferably from 1 mm to 30 mm and more preferably from 5 mm to 20 mm. In the nucleic acid extraction device 1100, if the length of the seventh plug 70 in the longitudinal direction of the tube part 100 is made longer, when the form in which the fourth plug 40 is ejected from the end of the tube part 100 at the fifth plug 50 side is employed, the sixth plug 60 may be made harder to be ejected. In this case, the specific length of the seventh plug 70 may be from 10 mm to 50 mm.

Further, the seventh plug 70 has a function of suppressing mixing of the second cleansing liquid (the sixth plug 60) and the eluate (the fourth plug 40) with each other. Furthermore, if the seventh plug 70 contains oil having the higher viscosity, when particles etc. are moved at the boundary surface between the second cleansing liquid (the sixth plug 60) and itself, “swab effect” by the oil may be improved. Thereby, when particles etc. are moved from the plug of the second cleansing liquid of the sixth plug 60 to the seventh plug 70 of the oil, the water-soluble components attached to the particles etc. are harder to be taken into the seventh plug 70 (oil).

According to the nucleic acid extraction device 1100, the particles etc. with adsorbed nucleic acid may be cleansed in the second plug 20 and the sixth plug 60. Thereby, the cleansing efficiency of the particles etc. may be further improved.

Further, in the nucleic acid extraction device 1100, a chaotropic agent may be contained in the first cleansing liquid of the second plug 20. For example, guanidinium hydrochloride is contained in the first cleansing liquid of the second plug 20, and thereby, the particles etc. may be cleansed while the adsorption of the nucleic acid adsorbed to the particles etc. is maintained or enhanced. The concentration when the guanidinium hydrochloride is contained in the second plug 20 may be e.g. from 3 mol/L to 10 mol/L, preferably from 5 mol/L to 8 mol/L. When the concentration of the guanidinium hydrochloride is within the range, the nucleic acid adsorbed to the particles etc. may be adsorbed more stably and other foreign materials may be cleansed.

When the second cleansing liquid of the sixth plug 60 is water or a buffer solution, in the second plug 20 (first cleansing liquid), the nucleic acid adsorbed to the particles etc. may be adsorbed more stably and cleansing may be performed, and, in the sixth plug 60 (second cleansing liquid), the particles etc. may be further cleansed while the chaotropic agent is diluted.

It would be readily understood that, even in the nucleic acid extraction device 1100 having the sixth plug 60 and the seventh plug 70 within the tube part 100, the above described tap, container, reservoir, etc. may be added to the configuration and the same advantages as those described above may be obtained.

### 2. Nucleic Acid Extraction Kit

FIG. 14 schematically shows an example of a nucleic acid extraction kit according to an embodiment. A nucleic acid extraction kit 2000 exemplified in FIG. 14 includes parts forming the major part of the above described nucleic acid extraction device. The same signs are assigned to the same configurations as those explained in the section of “1. Nucleic Acid Extraction Device” and the detailed explanation will be omitted.

The nucleic acid extraction kit 2000 of the embodiment includes a tube 200 in which a first plug 10 of an oil, a second plug 20 of a first cleansing liquid not miscible with the oil, a third plug 30 of an oil, a fourth plug 40 of an eluate not miscible with the oil, and a fifth plug 50 of an oil are provided inside in the order, and a container 120 that can be connected to the end of the tube 200 at the first plug 10 side with interiors communicating with each other.

The tube 200 takes a form in which both ends of the tube part 100 of the nucleic acid extraction device 1000 are opened, and has a tubular shape having a cavity inside in which a liquid may be passed in the longitudinal direction. The internal shape, outer shape, size, properties, materials, etc. of the tube 200 are the same as those of the tube part 100 of the nucleic acid extraction device 1000. The plugs provided inside of the tube 200 are the same as those provided in the tube part 100 of the nucleic acid extraction device 1000. Further, the ends of the tube 200 may be sealed by detachable taps 110. When the ends of the tube 200 are sealed by the taps 110, for example, storage and transfer of the nucleic acid extraction kit 2000 are easier. Further, in use of the tube 200, if the tap 110 seals the end of the tube 200 at the fifth plug 50 side, when the particles etc. are moved inside of the tube 200, the movements of the respective plugs within the tube 200 may be suppressed and cleansing and extraction may be further facilitated. In addition, the tap 110 is detachable and the end of the tube 200 at the fifth plug 50 side may be opened, and the eluate of the fourth plug 40 in which the nucleic acid is eluted is easily ejected from the end of the tube 200 at the fifth plug 50 side.

The container 120 is the same as the container 120 explained in the section of the nucleic acid extraction device 1000.

In the example of FIG. 14, the ends of the tube 200 are sealed by the detachable taps 110. Further, the nucleic acid extraction kit 2000 may include a lid 122 that detachably seals an opening 121 of the container 120, and the opening 121 of the container 120 may be sealed by the detachable lid 122. Furthermore, in the nucleic acid extraction kit 2000, part or all of the components of an adsorption liquid may be contained in the container 120.

In the nucleic acid extraction kit 2000, the container 120 may contain the adsorption liquid and magnetic particles. In this case, when a specimen is introduced into the container 120, the step of adsorbing the nucleic acid contained in the specimen to the magnetic particles may be performed in the container 120. Thereby, preparation of another container is not required and pretreatment of PCR may be performed more promptly. Further, in this case, the opening 121 of the container 120 may be sealed by the detachable lid 122 as appropriate. The magnetic particles will be described later in detail.

If the container 120 is a material having flexibility as described above, the container 120 connected to the tube 200 is deformed, and thereby, the interior of tube 200 may be pressurized. In this manner, when the eluate of the fourth plug 40 in which the nucleic acid is eluted is ejected from the end of the tube 200 at the fifth plug 50 side, pressure may be easily applied from the first plug 10 side of the tube 200. Thereby, the eluate may be easily dispensed to e.g. the reaction container for PCR or the like.

In the nucleic acid extraction kit 2000, in addition to the tube 200 and the container 120, other configurations including e.g. a tap, a lid, an instruction manual, a reagent, and a case may be contained. Further, here, the example in which the five plugs are provided within the tube 200 is shown, however, as described in the section of “1.6. Nucleic Acid Extraction Device”, it would be readily understood that the sixth plug 60, the seventh plug 70, and other plugs may be provided within the tube 200 (the tube part 100) as appropriate.

The nucleic acid extraction kit 2000 of the embodiment has the container 120 that can be connected to the end of the tube 200 at the first plug 10 side with interiors communicating with each other, and thus, if the particles etc. and the specimen are contained within the container 120, the nucleic acid may be adsorbed to the particles etc. and, if the container 120 is connected to the end of the tube 200 at the first plug 10 side, the particles etc. maybe easily introduced into the tube 200 from the first plug 10 side of the tube 200. Further, the nucleic acid extraction kit 2000 of the embodiment has the container 120, and thereby, the container 120 may be shaken and the liquid within the container 120 may be sufficiently stirred. Thereby, the nucleic acid may be promptly adsorbed to the particles etc.

The container 120 is connected to the tube 200, and thereby, the particles etc. with adsorbed nucleic acid are easily introduced from the end of the tube 200 at the first plug 10 side and moved to the fourth plug 40. Thereby, the extraction of the nucleic acid may be easily performed in an extremely short time. The nucleic acid extraction kit 2000 moves the particles etc. with adsorbed nucleic acid within the tube 200, and thereby, the eluate containing nucleic acid with higher purity may be obtained. Therefore, according to the nucleic acid extraction kit 2000, time and effort required for the pretreatment for PCR may be significantly reduced.

3. Nucleic Acid Extraction Method

The above described nucleic acid extraction device, nucleic acid extraction kit, their modified forms, and a nucleic acid extraction apparatus, which will be described later, may be suitably used for the nucleic acid extraction method of the embodiment. As below, as an example of the nucleic acid extraction method of the embodiment, a method using the above described nucleic acid extraction kit 2000 will be described.

The nucleic acid extraction method of the embodiment includes a step of introducing a specimen containing nucleic acid in the container 120 having flexibility and containing magnetic particles M and the adsorption liquid, a step of swinging the container 120 to allow the nucleic acid to adsorb to the magnetic particles M, a step of connecting the container 120 to the end of the tube 200 at the first plug 10 side in which the first plug 10 of the oil, the second plug 20 of the first cleansing liquid not miscible with the oil, the third plug 30 of the oil, the fourth plug 40 of the eluate not miscible with the oil, and the fifth plug 50 of the oil are provided in the order with the interior of the container 120 and the interior of the tube 200 communicating with each other, a step of moving the magnetic particles M from inside of the container 120 through the tube 200 to the position of the fifth plug 50 by application of a magnetic force, and a step of eluting the nucleic acid from the magnetic particles M in the eluate of the fourth plug 40.

In the nucleic acid extraction method of the embodiment, various kinds of particles (e.g. silica particles, polymer particles, magnetic particles, etc.) may be used as long as the particles may be allowed to adsorb the nucleic acid using the adsorption liquid and moved within the tube 200. However, in one embodiment of the nucleic acid extraction method to be described, the magnetic particles M containing magnetic materials that may adsorb the nucleic acid to particle surfaces are used. Note that, when the other particles etc. than the magnetic particles M are moved within the tube, e.g. the gravity force or a potential difference may be used.

In the nucleic acid extraction method of the embodiment, a material penetrating the magnetic force through the container 120 and the tube 200 is selected and the magnetic force is applied from outside of the container 120 and the tube 200, and thereby, the magnetic particles M are moved within the container 120 and the tube 200.

The specimen contains nucleic acid as a target. Hereinafter, it may be simply referred to as “target nucleic acid”. The target nucleic acid is e.g. DNA and RNA (DNA: Deoxyribonucleic Acid and/or RNA: Ribonucleic Acid). The target nucleic acid is extracted from the specimen by the nucleic acid extraction method of the embodiment and eluted in the eluate, and then, used as e.g. a template of PCR. The specimen includes blood, nasal cavity mucus, oral cavity mucus, or other various biological specimens.

3.1. Step of Introducing Specimen into Container

The step of introducing the specimen into the container 120 may be performed by, for example, attaching the specimen to a cotton swab, inserting the cotton swab from the opening 121 of the container 120, and immersing it in the adsorption liquid. Or, the specimen may be introduced from the opening 121 of the container 120 using a pipette or the like. Or, when the specimen is in a paste state or solid state, for example, it may be attached to the inner wall of the container 120 or put into the container from the opening 121 of the container 120 using a spoon, tweezers, or the like.

### 3.2. Step of Allowing Nucleic Acid to Adsorb to Magnetic Particles

The step of allowing the nucleic acid to adsorb is performed by swinging the container 120. The step may be performed more efficiently, if there is the lid 122 sealing the opening 121 of the container 120 and to seal the container 120 using the lid. Through the step, the target nucleic acid is adsorbed to the surfaces of the magnetic particles M by the action of the chaotropic agent. At the step, not only the target nucleic acid but also other nucleic acids than the target nucleic acid and protein may be adsorbed to the surfaces of the magnetic particles M.

As a method of swinging the container 120, an apparatus such as a vortex shaker may be used, or the container may be shaken by a hand of an operator. Or, the container 120 may be swung while a magnetic field is applied from outside using the magnetism of the magnetic particles M. The time for swinging the container 120 may be appropriately set. For example, when the approximate shape of the container 120 is a cylindrical shape having a diameter of about 20 mm and a height of about 30 mm, the container 120 is swung by shaking by hand for about ten seconds, and thereby, the materials are sufficiently stirred and the nucleic acid is adsorbed to the surfaces of the magnetic particles M.

### 3.3. Step of Connecting Container to Tube

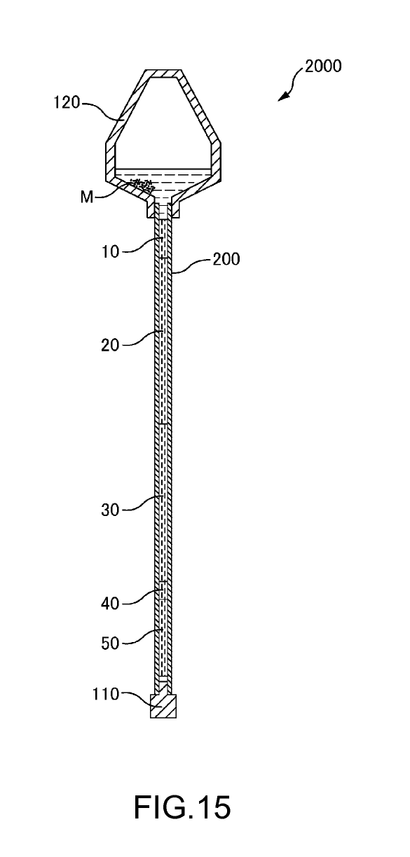

Then, as shown in FIG. 15, the container 120 is connected to the end of the tube 200 at the first plug 10 side. The respective plugs within the tube 200 are harder to move within the tube 200 even when the tap 110 at the first plug 10 side is detached because the tap 110 at the seventh plug 70 side is closed. The step is performed after the tap 110 is detached when the tap 110 is attached to the end of the tube 200 at the first plug 10 side. Then, the container 120 and the tube 200 are connected so that the contents may not leak out and the contents can communicate between the interior of the container 120 and the interior of the tube 200.

### 3.4. Step of Moving Magnetic Particles

Through the above described steps, the magnetic particles M with the adsorbed nucleic acid within the container 120 may pass to the tube 200. As a technique of guiding the magnetic particles M with the adsorbed nucleic acid to the tube 200, a method of using the gravity force or centrifugal force may be used, not particularly limited. In the embodiment, the step is performed by applying a magnetic force from outside of the container 120 and the tube 200. The magnetic force may be applied by e.g. a permanent magnet, an electromagnet, or the like, and it is preferable to use the permanent magnet for application because heat is not generated. Further, when the permanent magnet is used, the magnet may be moved by the hand of the operator or by using a mechanical device or the like. The magnetic particles M have properties to be attracted by the magnetic force, and moved from inside of the container 120 to the tube 200 by changing the relative position of the container 120 and the tube 200 and the permanent magnet using the properties. Thereby, the magnetic particles M are moved sequentially from the first plug 10 through the respective plugs to the fourth plug 40. The times in which the magnetic particles M stay in the respective plugs when the magnetic particles M pass through the respective plugs are not particularly limited, and the particles may be moved so as to reciprocate along the longitudinal direction of the tube 200 within the same plugs.

### 3.5. Step of Eluting Nucleic Acid

When the magnetic particles M reach the fourth plug 40, the nucleic acid adsorbed to the magnetic particles M is eluted in the eluate of the fourth plug 40 by the action of the eluate. Through the step, the nucleic acid is eluted from the specimen in the eluate and the nucleic acid is extracted from the specimen.

### 3.6. Advantages

According to the nucleic acid extraction method of the embodiment, the extraction of the nucleic acid may be easily performed in an extremely short time. The nucleic acid extraction method of the embodiment may obtain the eluate containing the nucleic acid with higher purity by moving the magnetic particles M with the adsorbed nucleic acid within the tube 200. According to the nucleic acid extraction method of the embodiment, time and effort required for the pretreatment for PCR may be significantly reduced.

3.7. Step of Ejecting Fourth Plug from Tube

The nucleic acid extraction method of the embodiment may include a step of ejecting the fifth plug 50 and the fourth plug 40 from the opposite end to the end to which the container 120 of the tube 200 is connected by deforming the container 120.

The step may be performed by deforming the container 120 after “3.5. Step of Eluting Nucleic Acid”. When the fourth plug 40 is ejected, the fifth plug 50 is ejected in advance. Note that the tap 110 sealing the fifth plug 50 side of the tube 200 is removed prior to the step and the end of the tube 20 at the fifth plug 50 side is opened.

When an external force is applied to the container 120 and the container is deformed so that the internal pressure may be increased, the respective plugs move from the first plug 10 side to the fifth plug 50 side of the tube 200 by the pressure. Thereby, the fifth plug 50 and the fourth plug 40 are sequentially ejected from the end of the tube 200 at the fifth plug 50 side. The third plug 30 (or the seventh plug 70) may be ejected, however, the second plug 20 (or the sixth plug 60) is not to be ejected. In this case, for example, the volume of the third plug 30 (or the seventh plug 70) is set to be larger than those of the other plugs and the length of the third plug 30 (or the seventh plug 70) in the longitudinal direction of the tube 200 is made larger, and thereby, the second plug 20 (or the sixth plug 60) is easily prevented from being ejected.

The fourth plug 40 and the fifth plug 50 are ejected into e.g. the reaction container for PCR. Accordingly, the eluate and the oil are dispensed in the reaction container for PCR, and generally, the oil does not affect the reaction of PCR and the reaction container of PCR may contain the same kind of oil as the oil of the fifth plug 50 in advance. Further, in this case, the step is performed in a state in which the end of the tube 200 is placed within the oil, and thereby, the eluate containing the target nucleic acid may be introduced into the reaction container of PCR without contact with the outside air. When the nucleic acid extraction method of the embodiment includes the step, the eluate containing the target nucleic acid may be easily dispensed into e.g. the reaction container for PCR or the like.

### 3.8. Modified Examples

### 3.8.1. Modification of Step of Moving Magnetic Particles

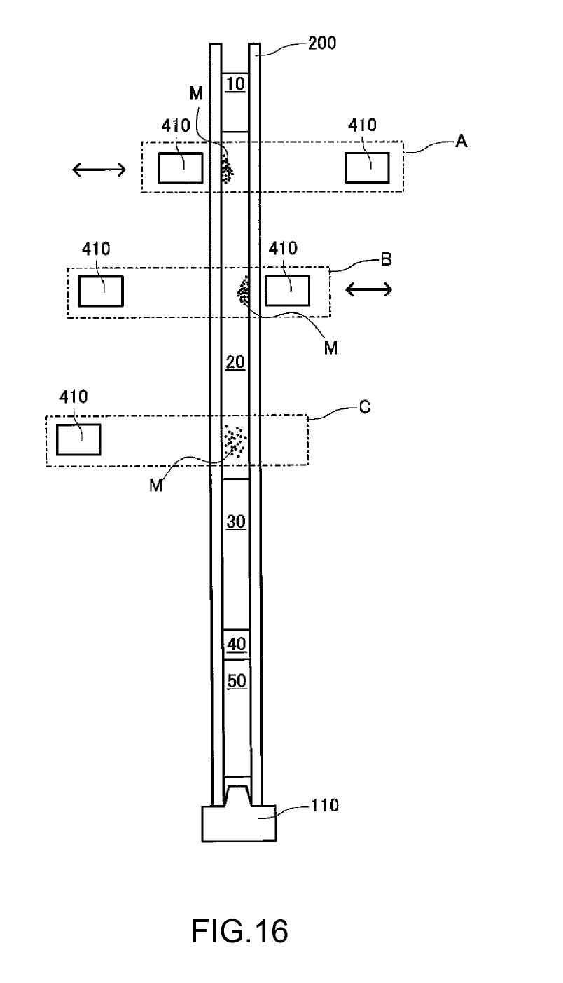

FIG. 16 is a schematic diagram for explanation of a modified example of the nucleic acid extraction method of the embodiment.

At the above described “3.4. Step of Moving Magnetic Particles”, the explanation that the magnetic particles Mare moved from the first plug 10 through the respective plugs to the fourth plug 40 by applying a magnetic force to the magnetic particles M from outside is made. However, when the magnetic particles M are moved to the second plug 20, the magnetic particles M may be vibrated within the second plug 20 and diffusion and agglomeration may be repeated by changing the magnetic force applied from outside. In this manner, the cleansing effect of the magnetic particles M by the first cleansing liquid of the second plug 20 may be improved.

Specifically, as shown by A and B in FIG. 16, in the case where a pair of permanent magnets 410 are used as means for applying a magnetic force, when the magnetic particles M are moved from the container 120 by the permanent magnets 410, passed through the first plug 10, and the magnetic particles M reach the second plug 20, one permanent magnet 410 is moved away from the tube 200 and the other permanent magnet 410 is moved closer to the tube 200 from the opposed side, and thereby, the magnetic particles M may be vibrated in directions intersecting with the longitudinal direction of the tube 200 within the second plug 20 (the forms of A, B in the drawing are repeated). In this manner, the cleansing effect of the magnetic particles M by the first cleansing liquid of the second plug 20 may be improved. The cleansing of the magnetic particles M may be applied, when the second plug 20 is divided and the sixth plug 60 is provided within the tube 200, to a plurality of the second plugs 20 and the sixth plug 60.

Further, as shown by C in FIG. 16, the permanent magnet 410 is simply moved away from the tube 200, and thereby, the magnetic particles M may be diffused within the second plug 20. The magnetic particles M have hydrophilic surfaces, and, for example, even when the magnetic force is weakened and the particles are diffused in the second plug 20, the particles are hard to enter the oils of the first plug 10 and the third plug 30. Therefore, the form may be employed.

Specifically, the magnetic particles M are moved from the container 120 by the permanent magnets 410, and, when the magnetic particles M are passed through the first plug 10 and reach the second plug 20, the permanent magnets 410 are moved away from the tube 200 and the magnetic particles M are diffused within the second plug 20. Then, the magnetic particles M may be moved by the magnetic force of the permanent magnets 410 again, passed through the third plug 30, and guided to the fourth plug 40.

The form of vibrating the magnetic particles M and repeating diffusion and agglomeration by changing the magnetic force applied from outside may be applied to a state in which the magnetic particles M exist in the adsorption liquid within the container 120 and a state in which the magnetic particles M exist in the fourth plug 40 (eluate).

### 3.8.2. Modification of Step of Eluting Nucleic Acid

The above described “3.5. Step of Eluting Nucleic Acid” may be performed by heating the fourth plug 40. As a method of heating the fourth plug 40, e.g. a method of bringing a heat medium such as a heat block into a position corresponding to the fourth plug 40 of the tube 200, a method of using a heat source such as a heater, a method by electromagnetic heating, etc. may be exemplified.

When the fourth plug 40 is heated, the other plugs than the fourth plug 40 may be heated. However, when the magnetic particles M with adsorbed nucleic acid exist in the plug of the cleansing liquid, it is preferable not to heat the plug. The temperature to reach when the fourth plug 40 is heated is preferably from 35° C. to 85° C., more preferably from 40° C. to 80° C., and even more preferably from 45° C. to 75° C. in view of elution efficiency and, if the eluate contains the enzyme of PCR, suppression of deactivation of the enzyme.

At the step of eluting the nucleic acid, when the fourth plug 40 is heated, the nucleic acid adsorbed to the magnetic particles M may be eluted in the eluate more efficiently. Further, even when the composition of the eluate is the same as or similar to that of the first cleansing liquid or the second cleansing liquid, the nucleic acid not eluted in the cleansing liquid, but left and adsorbed to the magnetic particles M may be eluted in the eluate. That is, even after the magnetic particles M with adsorbed nucleic acid are cleansed by the first cleansing liquid or the second cleansing liquid, the nucleic acid may be further eluted in the eluate. Thereby, even when the composition of the cleansing liquid and the composition of the eluate are the same or similar, both sufficient cleansing and elution with sufficient concentration in the eluate may be realized.

3.8.3. Modification of Step of Ejecting Fourth Plug from Tube

When the above described “3.7. Step of Ejecting Fourth Plug from Tube” is employed, at the step, the magnetic particles M that have eluted the adsorbed nucleic acid in the eluate may exist within the fourth plug 40. However, the step may be performed after the particles are moved into one of the first plug 10, the second plug 20, and the third plug 30 or the container 120 by application of a magnetic force. In this manner, the fourth plug 40 may be ejected from the tube 200 in the state in which the eluate does not contain the magnetic particles M. Further, the destination to which the magnetic particles M are moved is set to the second plug 20 or the container 120, and thereby, the magnetic particles M are hard to enter the oil of the third plug 30 even when the magnetic force is removed and the ejection of the fourth plug 40 from the tube 200 may be easily performed.

### 4. Nucleic Acid Extraction Apparatus

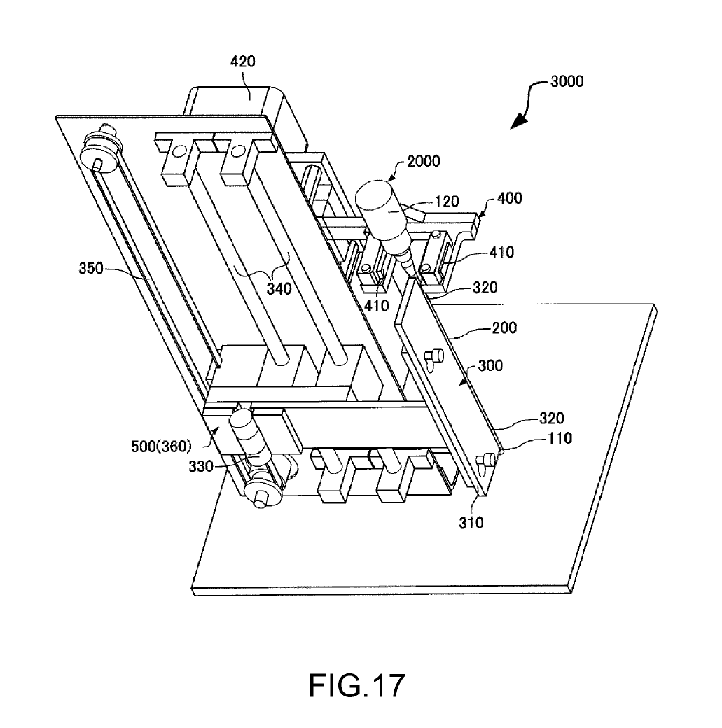

The nucleic acid extraction apparatus according to the embodiment may be suitably applied to the above described nucleic acid extraction device, nucleic acid extraction kit, nucleic acid extraction method. As below, a nucleic acid extraction apparatus 3000 that performs extraction of nucleic acid using the nucleic acid extraction kit 2000 attached thereto will be explained as one embodiment. FIG. 17 is a perspective view schematically showing an example of the nucleic acid extraction apparatus 3000 of the embodiment.

The nucleic acid extraction apparatus 3000 of the embodiment has a longitudinal direction, and includes an attachment part 300 in which a first plug 10 of an oil, a second plug 20 of a first cleansing liquid not miscible with the oil, a third plug 30 of an oil, a fourth plug 40 of an eluate not miscible with the oil, and a fifth plug 50 of an oil are provided in the order, a magnetic force application part 400 that applies a magnetic force from a side surface of a tube 200 when the tube 200 is attached to the attachment part 300, and a movement mechanism 500 that changes the relative position of the attachment part 300 and the magnetic force application part 400 along the longitudinal direction of the tube 200.

The tube 200 attached to the attachment part 300 of the nucleic acid extraction apparatus 3000 is the above described tube 200. The nucleic acid extraction apparatus 3000 has the attachment part 300 to which the tube 200 is attached. Note that the example in which the first plug 10 to the fifth plug 50 are provided within the tube 200 is described, however, the above described sixth plug 60 and seventh plug 70 may be provided.

The attachment part 300 is a part to which the tube 200 is attached. To the attachment part 300, a container 120 connected to the tube 200 may be attached with the tube 200. For the attachment part 300, a configuration, a mechanism for attachment, etc. within the range in which the magnetic force may be applied by the magnetic force application part 400 may be appropriately designed with respect to the tube 200 and the container 120 as appropriate. The attachment part 300 may be adapted, in the case where the tube 200 has flexibility and bends or the like, to extend the tube 200 into a linear shape and attach it. Further, in the illustrated example, the attachment part 300 has a doubling plate 310 provided so that the tube 200 may lie along the plate. The doubling plate 310 is not an essential configuration, however, if the doubling plate 310 is provided, vibration etc. of the tube 200 may be suppressed. Furthermore, in the illustrated example, the attachment part 300 has clip mechanisms 320, and thereby, the tube 200 is fixed in two positions.

The attachment part 300 is adapted to relatively change the position relationship with the magnetic force application part 400 with respect to the longitudinal direction of the tube 200. Therefore, when the attachment part 300 is designed to be moved relatively to the magnetic force application part 400 without movement of the magnetic force application part 400, the movement mechanism 500 includes a movement mechanism 360 that moves the attachment part 300 as shown in the drawing. Or, when the magnetic force application part 400 includes a movement mechanism, the movement mechanism 360 may be unnecessary for the attachment part 300. In the illustrated example, the attachment part 300 includes a hinge 330, guide rails 340, a drive belt 350, and a motor (not shown).

In the example of the nucleic acid extraction apparatus 3000, one attachment part 300 is provided, however, a plurality of the attachment parts may be provided. In this case, a plurality of the magnetic force application parts 400 may be provided, and the plurality of attachment parts 300 may be provided independently or interlocked.

The magnetic force application part 400 is adapted to apply the magnetic force to the tube 200 and the container 120 as appropriate when the tube 200 is attached to the attachment part 300. The magnetic force application part 400 includes e.g. a permanent magnet, an electromagnet, or a combination of them. The magnetic force application part 400 includes at least one magnet or the like and may include a plurality of magnets or the like. It is preferable to use, not the electromagnet, but the permanent magnet for the magnetic force application part 400 because heat or the like is not generated. As the permanent magnet, e.g. a nickel magnet, an iron magnet, a cobalt magnet, a samarium magnet, or a neodymium magnet may be used.

The magnetic force application part 400 has a function of applying the magnetic force to magnetic particles M existing within the container 120 and within the tube 200. Further, the relative position relationship between the attachment part 300 and the magnetic force application part 400 is changed, and thereby, the magnetic particles M may be moved within the container 120 and within the tube 200.

In the illustrated example, the magnetic force application part 400 has a pair of permanent magnets 410 provided to be opposed with the container 120 and the tube 200 in between. The pair of permanent magnets 410 are separated with a gap larger than the outer diameter of the tube 200. The direction of polarity of the permanent magnets 410 is not particularly limited. The magnetic force application part 400 is adapted to relatively change the position relationship with the attachment part 300 with respect to the longitudinal direction of the tube 200. Therefore, when the magnetic force application part 400 is designed to be moved relatively to the attachment part 300 without movement of the attachment part 300, the movement mechanism 500 includes a movement mechanism that moves the magnetic force application part 400.

Further, in the illustrated example, the magnetic force application part 400 is provided so that one of the pair of permanent magnets 410 may move closer to the tube 200 and the other may move away from the tube 200. By a motor 420, the pair of permanent magnets 410 may be vibrated to move closer to and away from the tube 200. The motor 420 is driven, and thereby, the magnetic particles M may be reciprocated in directions intersecting with the longitudinal direction of the tube 200 within the tube 200.

The motor 420 may be driven as appropriate regardless of the position of the container 120 or the tube 200 to which the magnetic force is applied. However, if the motor is driven when the positions of the permanent magnets 410 are located in the positions of the second plug 20 and the fourth plug 40 of the tube 200, the cleansing efficiency and the elution efficiency of the magnetic particles M within the tube 200 may be improved.

According to the nucleic acid extraction apparatus 3000 of the embodiment, pretreatment for PCR can be automated and time and effort required for the pretreatment may be significantly reduced. Further, according to the nucleic acid extraction apparatus 3000 of the embodiment, the magnetic force application part 400 may be swung, and thereby, cleansing (purification) of the magnetic particles M with adsorbed nucleic acid may be performed more efficiently and the accuracy of PCR may be further improved.

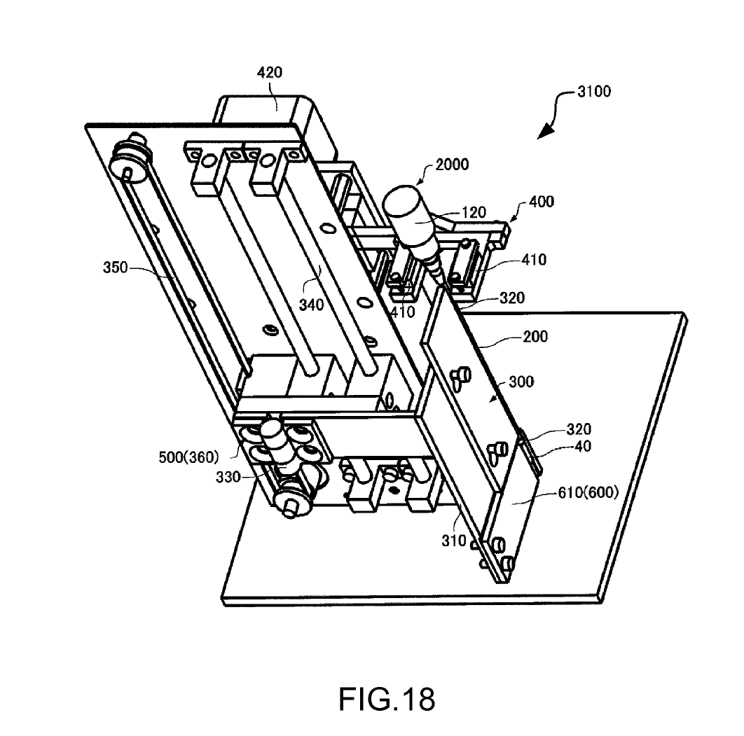

FIG. 18 is a perspective view schematically showing a nucleic acid extraction apparatus 3100 according to a modified example of the nucleic acid extraction apparatus. The nucleic acid extraction apparatus 3100 is the same as the above described nucleic acid extraction apparatus 3000 except the difference in having a heating part 600, and the same signs are assigned to the members having actions and functions in common and their explanation will be omitted.

The heating part 600 has a configuration that heats a part of the tube 200 when the tube 200 is attached to the attachment part 300. As the heating part 600, e.g. a heat source and a heat block, a heater, a coil for electromagnetic heating, or the like may be exemplified. As the shape of the heating part 600, any shape such as a shape into which the tube 200 maybe inserted and a shape in contact with the side surface of the tube 200 may be employed as long as the liquid within the tube 200 may be heated.

The portion of the tube 200 heated by the heating part 600 includes a portion in which the fourth plug 40 exists in the longitudinal direction of the tube 200. The heating part 600 may heat another portion of the tube 200, and it is preferable not to heat a portion in which the second plug 20 exists in the longitudinal direction of the tube 200.

In the nucleic acid extraction apparatus 3100 shown in FIG. 18, a heater 610 that heats the position including the fourth plug 40 of the tube 200 is provided as the heating part 600 in parallel to the doubling plate 310. The heater 610 has a shape in contact with about a half of the outer circumference of the tube 200.

Even when the amount of the nucleic acid adsorbed to the magnetic particles M is reduced by the cleansing by at least one of the first cleansing liquid of the second plug 20 and the second cleansing liquid of the sixth plug 60, the nucleic acid extraction apparatus 3100 may elute a sufficient amount of nucleic acid in the eluate of the fourth plug 40. Thereby, the cleansing effect may be improved and the nucleic acid with sufficient concentration for PCR may be eluted in the eluate.

### 5. Experimental Examples

As below, experimental examples will be explained and the invention will be explained further in detail, however, the invention is not limited to the following experimental examples.

### 5.1. Experimental Example 1

In the experimental example 1, of the above described nucleic acid extraction kits 2000, the configuration having the first plug 10 to the seventh plug 70 within the tube 200 was used.

First, in a polyethylene container having a volume of 3 mL, 375 μL of an adsorption liquid and 1 μL of a magnetic bead dispersion liquid were put. As the composition of the adsorption liquid, 76% by mass of guanidinium hydrochloride, 1.7% by mass of ethylenediaminetetraacetic acid disodium salt dehydrate, and a water solution of 10% by mass of polyoxyethylene sorbitan monolaurate (manufactured by Toyobo, MagExtractor-Genome-, NPK-1). As the magnetic bead dispersion liquid, a liquid containing 50% by volume of magnetic silica particles and 20% by mass of lithium chloride was used.

50 μL of blood obtained from a human was put from the opening of the container using a pipette, the container was closed by a lid and shaken by hand for thirty seconds and stirred. Then, the lid of the container was detached and the container was connected to a tube. Note that both ends of the tube had been closed by taps, and the tap at the first plug side was detached and the container was connected to the tube.

Here, the first, third, seventh, fifth plugs contained silicon oil. The first cleansing liquid of the second plug was a water solution of 76% by mass of guanidinium hydrochloride. Further, the second cleansing liquid of the sixth plug was Tris-HCL buffer solution (solute concentration of 5 mM) with a pH of 8.0. The eluate of the fourth plug was sterilized water.

Then, the permanent magnets were moved by hand and the magnetic beads within the container were introduced into the tube. Then, the magnetic beads were moved to the fourth plug. The times in which the magnetic beads exist in the respective plugs within the tube were approximately as follows. The first, third, seventh plugs: respective three seconds, the second plug: 20 seconds, the sixth plug: 20 seconds and the fourth plug: 30 seconds. In the second plug and the sixth plug, operation of vibrating the magnetic beads or the like was not performed. Further, the volumes of the second plug, the sixth plug, and the fourth plug were 25 μL, 25 μL, 1 μL, respectively.

Subsequently, the tap at the fifth plug side of the tube was detached, the container was deformed by hand, and the fifth plug and the fourth plug were ejected into the reaction container of PCR. The operation was performed after the magnetic beads were moved by the permanent magnets and retracted to the second plug.

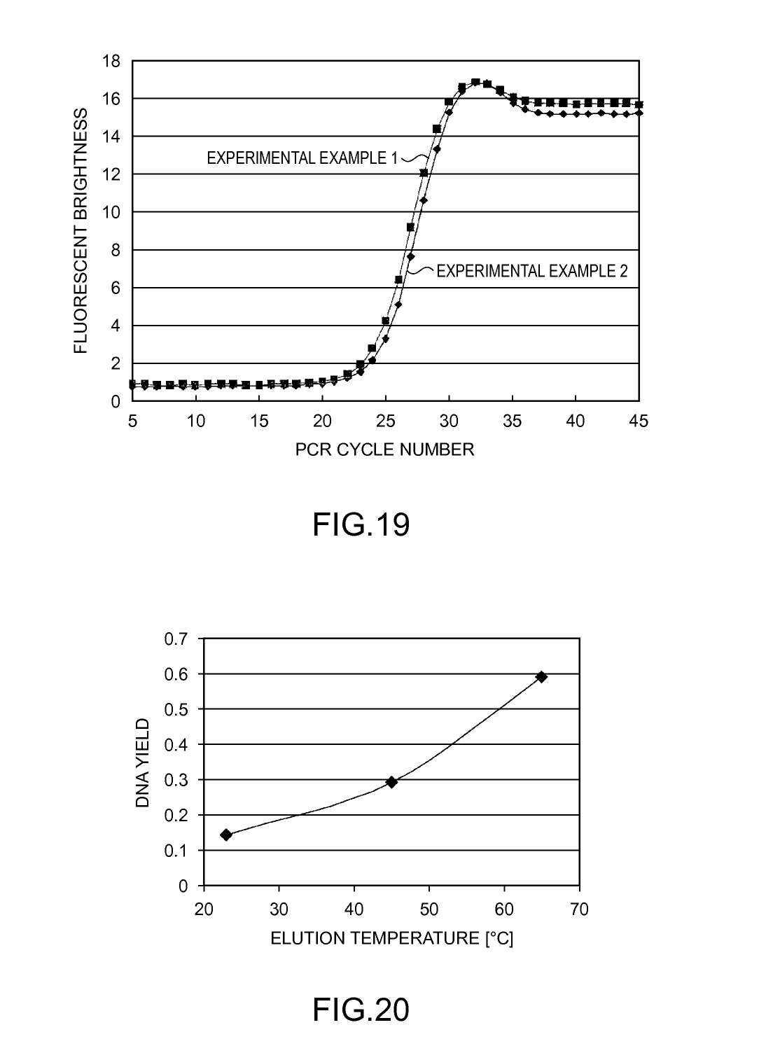

Then, 19 μL of a reaction reagent of PCR was added to the extraction liquid, and real-time PCR was performed according to the usual method. The contents of the reaction reagent of PCR were 4 μL of LightCycler 480 Genotyping Master (manufactured by Roche Diagnostics 4 707 524), 0.4 μL of 1000-fold diluted SYBR Green I (manufactured by Lifetechnologies 57563) by sterilized water, respective 0.06 μL of 100 μM of β-actin detection primers (F/R), and 14.48 μL of sterilized water. FIG. 19 shows an amplification curve of PCR of the experimental example 1. Note that the vertical axis of FIG. 19 indicates fluorescent brightness, and the horizontal axis indicates PCR cycle number.

### 5.2. Experimental Example 2

In the experimental example 2, extraction of nucleic acid was performed using a general nucleic acid extraction method.

First, in a polyethylene container having a volume of 1.5 mL, 375 μL of an adsorption liquid and 20 μL of a magnetic bead dispersion liquid were put. The compositions of the adsorption liquid and the magnetic bead dispersion liquid are the same as those of the experimental example 1.

Then, 50 μL of blood obtained from a human was put from the opening of the container using a pipette, the container was closed by a lid and stirred for ten minutes by a vortex mixer, and B/F separation operation was performed by operating a magnetic stand and a pipette. In the state, in the container, the magnetic beads and a small amount of the adsorption liquid were left.

Then, 450 μL of the first cleansing liquid having the same composition as that of the experimental example 1 was introduced into the container, the container was closed by the lid and stirred for five seconds by the vortex mixer, and the first cleansing liquid was removed by operating the magnetic stand and the pipette. The operation was repeated twice. In the state, in the container, the magnetic beads and a small amount of the first cleansing liquid were left.

Then, 450 μL of the second cleansing liquid having the same composition as that of the experimental example 1 was introduced into the container, the container was closed by the lid and stirred for five seconds by the vortex mixer, and the second cleansing liquid was removed by operating the magnetic stand and the pipette. The operation was repeated twice. In the state, in the container, the magnetic beads and a small amount of the second cleansing liquid were left.

Then, 50 μL of sterilized water (eluate) was added in the container, the container was closed by the lid and stirred for ten minutes by the vortex mixer, and a supernatant liquid was collected by operating the magnetic stand and the pipette. The supernatant liquid contained target nucleic acid.

Then, 1 μL was dispensed from the extraction liquid, 19 μL of a reaction reagent of PCR was added, and real-time PCR was performed according to the usual method. The contents of the reaction reagent of PCR were 4 μL of LightCycler 480 Genotyping Master (manufactured by Roche Diagnostics 4 707 524), 0.4 μL of 1000-fold diluted SYBR Green I (manufactured by Lifetechnologies 57563) by sterilized water, respective 0.06 μL of 100 μM of β-actin detection primers (F/R), and 14.48 μL of sterilized water. FIG. 19 shows an amplification curve in this regard.

### 5.3. Experimental Example 3

In the experimental example 3, of the above described nucleic acid extraction kits 2000, the configuration having the first plug 10 to the fifth plug 50 within the tube 200 was used.

The compositions of the adsorption liquid and the magnetic bead dispersion liquid were the same as those of the experimental example 1, and the first, third, fifth plugs were silicon oil as is the case of the experimental example 1.

The first cleansing liquid of the second plug was Tris-HCL buffer solution (solute concentration 5 mM) with a pH of 8.0. The eluate of the fourth plug was sterilized water.

50 μL of blood obtained from a human was put from the opening of the container using a pipette, the container was closed by a lid and shaken for thirty seconds by hand and stirred. Then, the lid of the container was detached and the container was connected to a tube. Note that both ends of the tube had been closed by taps, and the tap at the first plug side was detached and the container was connected to the tube.

Then, the permanent magnets were moved by hand and the magnetic beads within the container were introduced into the tube. Then, the magnetic beads were moved to the fourth plug. The times in which the magnetic beads exist in the respective plugs within the tube were approximately as follows. The first, third plugs: respective three seconds, the second plug: 20 seconds, and the fourth plug: 30 seconds. In the second plug, operation of vibrating the magnetic beams or the like was not performed. Further, the volumes of the second plug and the fourth plug were 25 μL and 1 μL, respectively.

Subsequently, the tap at the fifth plug side of the tube was detached, the container was deformed by hand, and the fifth plug and the fourth plug were ejected into the reaction container of PCR. The operation was performed after the magnetic beads were moved by the permanent magnets and retracted to the second plug.

Then, 19 μL of a reaction reagent of PCR was added to the extraction liquid, and real-time PCR was performed according to the usual method. The contents of the reaction reagent of PCR were 4 μL of LightCycler 480 Genotyping Master (manufactured by Roche Diagnostics 4 707 524), 0.4 μL of 1000-fold diluted SYBR Green I (manufactured by Lifetechnologies 57563) by sterilized water, respective 0.06 μL of 100 μM of β-actin detection primers (F/R), and 14.48 μL of sterilized water.

An amplification curve in this regard has nearly the same characteristics as those in FIG. 19. Note that, when the same experiment was conducted with the first cleansing liquid of the second plug of 76% by mass of guanidinium hydrochloride in the experimental example, ten or more cycles of delay in rising from the amplification curve of the experimental example 1 appeared.

### 5.4. Experimental Example 4

### Influence by Elution Temperature on DNA Yield

In the experimental example 4, extraction of nucleic acid was performed using a general nucleic acid extraction method.

First, in a polyethylene container having a volume of 1.5 mL, 375 μL of an adsorption liquid and 20 μL of a magnetic bead dispersion liquid were put. The compositions of the adsorption liquid and the magnetic bead dispersion liquid are the same as those of the experimental example 1.

Then, 50 μL of a genome DNA solution prepared with concentration of 1 ng/μL was introduced from the opening of the container using a pipette, the container was closed by a lid and stirred for ten minutes by a vortex mixer, and B/F separation operation was performed by operating a magnetic stand and a pipette. In the state, in the container, the magnetic beads and a small amount of the adsorption liquid were left.

Then, 450 μL of the first cleansing liquid having the same composition as that of the experimental example 1 was introduced into the container, the container was closed by the lid and stirred for five seconds by the vortex mixer, and the first cleansing liquid was removed by operating the magnetic stand and the pipette. The operation was repeated twice. In the state, in the container, the magnetic beads and a small amount of the first cleansing liquid were left.

Then, 450 μL of the second cleansing liquid having the same composition as that of the experimental example 1 was introduced into the container, the container was closed by the lid and stirred for five seconds by the vortex mixer, and the second cleansing liquid was removed by operating the magnetic stand and the pipette. The operation was repeated twice. In the state, in the container, the magnetic beads and a small amount of the second cleansing liquid were left.

Then, 50 μL of sterilized water (eluate) is added in the container, the container was closed by the lid and stirred for five seconds by the vortex mixer, and then, heated for two minutes using a tube heater. Then, the container was stirred again for ten seconds by the vortex mixer, and a supernatant liquid was collected by operating the magnetic stand and the pipette. The heating temperatures by the tube heater were set to three temperatures of 23° C. (left at the room temperature), 45° C., 65° C.

Then, 1 μL was dispensed from the extraction liquid, 19 μL of a reaction reagent of PCR is added, and real-time PCR was performed according to the usual method. Concurrently, as a comparison sample, a genome DNA solution prepared with concentration of 1 ng/μL was added to PCR reaction samples. The contents of the reaction reagent of PCR were 4 μL of LightCycler 480 Genotyping Master (manufactured by Roche Diagnostics 4 707 524) , 0.4 μL of 1000-fold diluted SYBR Green I (manufactured by Lifetechnologies S7563) by sterilized water, respective 0.06 μL of 100 μM of β-actin detection primers (F/R), and 14.48 μL of sterilized water.

FIG. 20 shows a relationship between the elution temperature and the DNA yield in this regard. The result was obtained by calculation from the rising cycle of the real-time PCR. Suppose that the rising cycle of the comparison sample is Ct0 and the rising cycle of the extraction sample is Ct1, the DNA yield is expressed by expression “2^{(Ct0-Ct1)}” as a ratio of the comparison sample (=1).

### 5.5. Experimental Example 5

In the experimental example 5, influences by the distance from the plug of the nucleic acid extraction device to the hand wearing the nitrile glove as the charged material on displacement and disruption of the plug within the tube were researched.

First, ten polypropylene tubes having inner diameters of 1 mm and outer diameters of 3 mm were prepared. Each tube is filled with silicone oil having kinetic viscosity of 2 cSt (25° C.) and 1 μL of pure water. Then, the portion filled with the silicone oil and the water of each tube was grasped by the hand wearing the nitrile glove, and the hand was vertically reciprocated at ten times. Then, the number of tubes with the level of the water displaced by 1 mm or more and the number of tubes with disrupted water were counted. As a result, in all of the ten tubes, displacement of the water level and disruption of the plug occurred.

Then, ten polypropylene tubes having inner diameters of 3 mm and outer diameters of 5 mm and ten polypropylene tubes having inner diameters of 1 mm and outer diameters of 3 mm were respectively prepared. Then, the polypropylene tubes having inner diameters of 1 mm and outer diameters of 3 mm were inserted into the respective inner cavities of the polypropylene tubes having inner diameters of 3 mm and outer diameters of 5 mm, and ten tubes having the inner diameters of 1 mm and the outer diameters of 5 mm were obtained.

Each tube is filled with silicone oil having kinetic viscosity of 2 cSt (25° C.) and 1 μL of pure water. Then, the portion filled with the silicone oil and the water of each tube was grasped by the hand wearing the nitrile glove, and the hand was vertically reciprocated at ten times. Then, the number of tubes with the level of the water displaced by 1 mm or more and the number of tubes with disrupted water were counted. As a result, in nine of the ten tubes, displacement of the water level and disruption of the plug occurred.

Then, ten polypropylene tubes having inner diameters of 5 mm and outer diameters of 7 mm, ten polypropylene tubes having inner diameters of 3 mm and outer diameters of 5 mm, and ten polypropylene tubes having inner diameters of 1 mm and outer diameters of 3 mm were respectively prepared. Then, the polypropylene tubes having inner diameters of 3 mm and outer diameters of 5 mm were inserted into the respective inner cavities of the polypropylene tubes having inner diameters of 5 mm and outer diameters of 7 mm, the polypropylene tubes having inner diameters of 1 mm and outer diameters of 3 mm were inserted into the respective inner cavities of the polypropylene tubes having inner diameters of 3 mm and outer diameters of 5 mm, and ten tubes having the inner diameters of 1 mm and the outer diameters of 7 mm were obtained.

Each tube is filled with silicone oil having kinetic viscosity of 2 cSt (25° C.) and 1 μL of pure water. Then, the portion filled with the silicone oil and the water of each tube was grasped by the hand wearing the nitrile glove, and the hand was vertically reciprocated at ten times. Then, the number of tubes with the level of the water displaced by 1 mm or more and the number of tubes with disrupted water were counted. As a result, in none of the ten tubes, displacement of the water level and disruption of the plug occurred.

### 5.6. Experimental Results

The following things were turned out from the above described experimental examples.

(1) In comparison between times taken for extraction treatment of nucleic acid as pretreatment of PCR, the time from insertion of the specimen into the container to introduction of the target nucleic acid into a reaction container of PCR was about two minutes in the experimental example 1. It was about thirty minutes in the experimental example 2. Thereby, it has been found that, in the nucleic acid extraction method of the experimental example 1, the time taken for nucleic acid extraction is significantly shorter than that in the nucleic acid extraction method of the experimental example 2.

(2) Further, the amounts of the respective cleansing liquids in the experimental example 1 were about one eighteenth of the amounts in the experimental example 2. Furthermore, the amount of the eluate in the experimental example 1 is about one fiftieth of the amount in the experimental example 2. Therefore, in the experimental example 1, it has been turned out that the very small amounts of the cleansing liquids and the eluate are sufficient in the experimental example 1 compared to those in the experimental example 2.
(3) In comparison between the concentrations of the target nucleic acid in the eluate in the amounts of the adsorption liquid and the eluate, it is considered that, ideally, the concentration in the experimental example 1 is fifty times the concentration in the experimental example 2. Note that, in the above described experimental examples, the amount of nucleic acid contained in the blood sample is large and exceeds the amount that can be adsorbed by 1 μL of the magnetic beads, and the whole amount of the nucleic acid contained in the blood sample can hardly be collected and the concentration fifty times the concentration in the experimental example 2 was not obtained in the experimental example 1. In the case of a specimen having a smaller nucleic acid content not exceeding the amount that can be adsorbed by 1 μL of the magnetic beads, the concentration fifty times the concentration in the experimental example 2 is obtained in the experimental example 1.

(4) From the graph in FIG. 19, it has been turned out that, even in a whole blood sample having a larger nucleic acid content, the rising of the amplification factor of nucleic acid is earlier by about 0.6 cycle in the experimental example 1 than that in the experimental example 2. Namely, it has been turned out that the reaction liquid of PCR used in the experimental example 1 has the higher concentration of the target nucleic acid than that of the reaction liquid of PCR used in the experimental example 2. Thereby, it has been supported that the concentration of the target nucleic acid in the eluate is higher in the experimental example 1 than that in the experimental example 2.

(5) From the result of the experimental example 3, it has been turned out that the extraction is sufficiently performed even when the second plug is the buffer. Further, it has been turned out that, when the second plug is the guanidinium water solution, the rising of the PCR amplification curve significantly delays due to the influence by enzyme reaction inhibition. Furthermore, it has been turned out that the influence by enzyme reaction inhibition on the guanidinium water solution may be suppressed by dilution of the extraction liquid to at least 1000 times.

(6) From the result of the experimental example 4, it has been turned out that the yield of DNA becomes sufficient for use for PCR by setting the fourth plug to be higher than about 40° C.

(7) From the result of the experimental example 5, it has been turned out that the charged material is separated at 3 mm or more from the liquid part filled with the reagent, i.e., the plug portion, and thereby, displacement of the liquid level and disruption of the plug may be suppressed.

The invention is not limited to the above described embodiments, and other various modifications may be made. For example, the invention includes substantially the same configurations as the configurations explained in the embodiments (e.g. the configurations with the same functions, methods, and results or the configurations with the same purposes and effects). Further, the invention includes configurations in which non-essential parts of the configurations explained in the embodiments are replaced. Furthermore, the invention includes configurations providing the same advantages or configurations that may reach the same purposes as those of the configurations explained in the embodiments. In addition, the invention includes configurations formed by adding known technologies to the configurations explained in the embodiments.

The entire disclosure of Japanese Patent Application No. 2014-239976, filed Nov. 27, 2014 is expressly incorporated by reference herein.

What is claimed is:

1. A nucleic acid extraction device comprising:
a tube part in which a first plug of an oil, a second plug of a cleansing liquid not miscible with the oil for cleansing a material with adsorbed nucleic acid, a third plug of an oil, a fourth plug of an eluate not miscible with the oil for eluting nucleic acid from the material, and a fifth plug of an oil are provided in this order; and
a cover part provided to surround the tube part.

2. A nucleic acid extraction device comprising:
a tube part in which a first plug of an oil and a second plug of a water solution not miscible with the oil are provided; and
a cover part provided to surround the tube part.

3. The nucleic acid extraction device according to claim 1, wherein the cover part is detachable.

4. The nucleic acid extraction device according to claim 1, wherein the cover part is extendable in a direction in which the tube part extends.

5. The nucleic acid extraction device according to claim 1, wherein the tube part and the cover part are separated.

6. The nucleic acid extraction device according to claim 1, wherein a distance from an inner cavity surface of the tube part and an outer surface of the cover part is equal to or more than 3 mm.

7. The nucleic acid extraction device according to claim 1, wherein the cover part is provided with a slit extending in a direction in which the tube part extends.

8. The nucleic acid extraction device according to claim 1, wherein the cover part is provided with a hole.

9. The nucleic acid extraction device according to claim 1, wherein the cover part is formed using a deformable material, and a gas is enclosed in between the tube part and the cover part and contact between the tube part and the cover part is prevented.

10. The nucleic acid extraction device according to claim 1, wherein the cover part contains a non-magnetic material selected from metals or alloys.

11. A nucleic acid extraction device comprising a tube part in which a first plug of an oil, a second plug of a cleansing liquid not miscible with the oil for cleansing a material with adsorbed nucleic acid, a third plug of an oil, a fourth plug of an eluate not miscible with the oil for eluting nucleic acid from the material, and a fifth plug of an oil are provided in this order,
wherein a thickness of a side wall of the tube part is equal to or more than 3 mm.

12. The nucleic acid extraction device according to claim 1, wherein a side wall of the tube part contains a non-magnetic material selected from metals or alloys.
